(19) United States
(12) Patent Application Publication
(10) Pub. No.: US 2025/0229081 A1
(43) Pub. Date: Jul. 17, 2025

(54) NANOMATERIAL COATED ELECTRODE AND METHODS OF USE THEREOF

(71) Applicant: University of Connecticut, Farmington, CT (US)

(72) Inventors: Yupeng Chen, Sharon, MA (US); Martin Han, Willington, CT (US); Ian Sands, Manchester, CT (US)

(21) Appl. No.: 18/698,254

(22) PCT Filed: Oct. 3, 2022
(86) PCT No.: PCT/US2022/045543

§ 371 Date: Apr. 3, 2024

Related U.S. Application Data
(60) Provisional application No. 63/251,841, filed on Oct. 4, 2021.

(51) Int. Cl. A61N 1/04; A61N 1/05

(52) U.S. Cl. CPC A61N 1/0496; A61N 1/0536

(57) ABSTRACT

Disclosed herein are electrically conductive coatings based on DNA-inspired Janus base nanotubes (JBNTs) for electrodes, microelectrodes, or macroelectrodes, as well as apparatuses and devices including the same, and methods of preparing and using the same.

### CROSS-REFERENCE TO RELATED APPLICATIONS

This application claims the priority benefit of U.S. Provisional Application No. 63/251,841, filed on 4 Oct. 2022, the entire contents of which is incorporated herein by reference.

### STATEMENT OF GOVERNMENT SUPPORT

This invention was made with government support under W81XWH-21-1-0274 awarded by the Medical Research and Materiel Command, AR072027 awarded by the National Institutes of Health, and 1905785, and 2025362 awarded by the National Science Foundation. The government has certain rights in the invention.

### BACKGROUND

Microelectrodes can be used in a variety of tissues, such as human tissues. One of the most promising applications is for brain-machine interfaces (BMI). Microelectrode coatings have been developed to facilitate the integration of brain and device, such as conductive polymers, carbon nanotubes, and bioactive hydrogels. However, many of these materials possess biological, functional, or electrochemical disadvantages that impede microelectrode efficacy, including bioincompatibility, cytotoxicity, or impede electrical conductivity. Accordingly, new microelectrode coatings are needed that have improved biocompatibility, cytotoxicity, and electrical conductivity to increase their function and lifetime of these microelectrodes in tissues, such as human tissue, for improved BMI and stimulation applications.

### SUMMARY

Disclosed herein are electrically conductive coatings based on DNA-inspired Janus base nanotubes (JBNTs) for electrode and insulation areas on electrodes (e.g., microelectrodes and macroelectrodes).

An aspect of the present disclosure relates to an electrode (e.g., microelectrode or macroelectrode) comprising a nanomaterial coating disposed on the electrode, wherein the coating comprises a Janus base nanotube. In any aspect or embodiment described herein, the electrode further comprises insulation areas disposed on the electrodes. In any aspect or embodiment described herein, the electrode is a microelectrode. In any aspect or embodiment described herein, the electrode is a macroelectrode.

In any aspect or embodiment described herein, the nanomaterial coating further comprises one or more therapeutic molecules. For example, in any aspect or embodiment described herein, the one or more therapeutic molecules are selected from Transforming Growth Factor-β (TGFβ), Vascular Endothelial Growth Factor (VEGF), an Insulin Like Growth Factor (IGF), an Epidermal Growth Factor (EGF), a Platelet-Derived Growth Factor (PDGF), a Bone Morphogenetic Protein (BMP), a Fibroblast Growth Factor (FGF), Glial Cell Derived Neurotrophic Factor (GDNF), Hepatocyte Growth Factor (HGF), Placental Growth Factor (PGF), Nerve Growth Factor (NGF), Tumor Necrosis Factor-α (TNF-α), Stromal Cell-Derived Factor 1 9SDF-1), dexamethasone, resveratrol a small interfering ribonucleic acid (siRNA), a micro ribonucleic acid (miRNA), a growth factor, a small-molecule drug, the like, and mixtures thereof.

In any aspect or embodiment described herein, the nanomaterial coating is a self-assembled nanomaterial coating. In any aspect or embodiment described herein, the nanomaterial coating is a single compartment nanomaterial coating. In any aspect or embodiment described herein, the nanomaterial coating is a multiple compartment nanomaterial coating.

Another aspect of the present disclosure relates to an apparatus or device comprising the electrode (e.g., a microelectrode or a macroelectrode) of the present disclosure. For example, an aspect of the present disclosure relates to an apparatus or device comprising an electrode (e.g., a microelectrode or macroelectrode), wherein the electrode comprises a nanomaterial coating disposed on the electrode, wherein the coating comprises a Janus base nanotube.

In any aspect or embodiment described herein, the electrode (e.g., microelectrode or macroelectrode) coatings of the present disclosure are electrically conductive coating based on DNA-inspired Janus base nanotubes (JBNTs). In any aspect or embodiment described herein, the JBNTS comprise a compound selected from a compound of Formula (I), (II), (III), (IV), (V), (VI), (VII), (VIII), (IX), (X), (XI), and combinations thereof.

In any aspect or embodiment described herein, the Janus base is a compound of Formula (I),

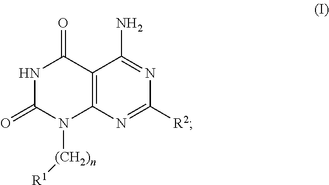

or a pharmaceutically acceptable salt thereof, wherein:
n is 1, 2, 3, 4, 5, or 6;
R¹ is selected from an α-amino acid, a β-amino acid, an α-polypeptide, and a β-polypeptide;
R² is selected from H, CH₃, and NHR^{z}; and
R^{z} is independently H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (III),

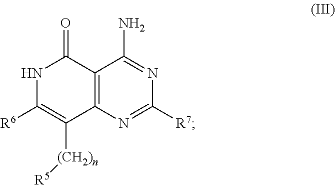

or a pharmaceutically acceptable salt thereof, wherein:
n is 1, 2, 3, 4, 5, or 6;
R⁵ is selected from an α-amino acid, a β-amino acid, an α-polypeptide, and a β-polypeptide;
each of R⁶ and R⁷ is independently selected from H, CH₃, and NHR^{z}; and
R^{z} is independently H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (V):

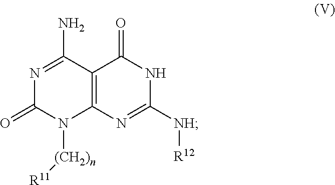

or a pharmaceutically acceptable salt or ester thereof, wherein:
n is 1, 2, 3, 4, 5, or 6;
R¹¹ is selected from an α-amino acid, a β-amino acid, an α-polypeptide, and a β-polypeptide; and
R¹² is H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (VII):

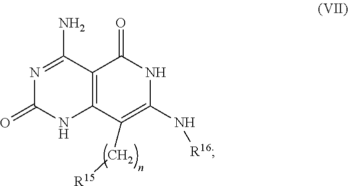

or a pharmaceutically acceptable salt or ester thereof, wherein:
n is 1, 2, 3, 4, 5, or 6;
R¹⁵ is selected from an α-amino acid, a β-amino acid, an α-polypeptide, and a β-polypeptide; and
R¹⁶ is H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (II):

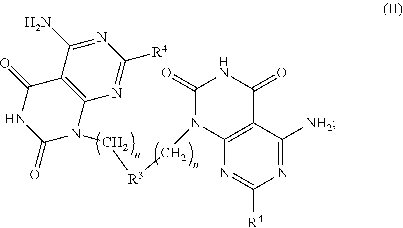

or a pharmaceutically acceptable salt thereof, wherein:
each n is independently 1, 2, 3, 4, 5, or 6;
R³ is selected from an α-amino acid, a β-amino acid, an α-polypeptide, and a β-polypeptide;
each of R⁴ is independently H, CH₃, or NHR^{z}; and
each R^{z} is H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (IV):

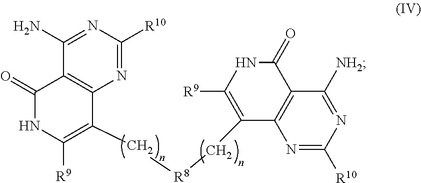

or a pharmaceutically acceptable salt thereof, wherein
each n is independently 1, 2, 3, 4, 5, or 6;
R⁸ is selected from an α-amino acid, a β-amino acid, an α-polypeptide, and a β-polypeptide;
each R⁹ and R¹⁰ is independently H, CH₃, or NHR^{z}; and
each R^{z} is H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (VI):

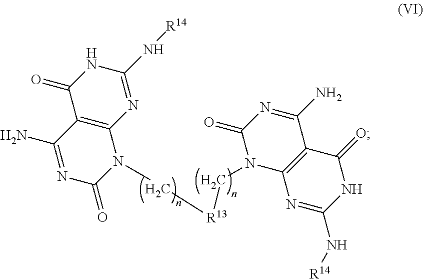

or a pharmaceutically acceptable salt thereof, wherein
each n is independently 1, 2, 3, 4, 5, or 6;
R¹³ is selected from an α-amino acid, a β-amino acid, an α-polypeptide, and a β-polypeptide; and
each R¹⁴ is independently H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (VIII):

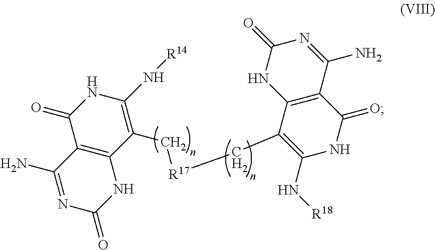

or a pharmaceutically acceptable salt thereof, wherein:
each n is independently 1, 2, 3, 4, 5, or 6;
R¹⁷ is selected from an α-amino acid, a β-amino acid, an α-polypeptide, and a β-polypeptide; and
each R¹⁸ is independently H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (IX):

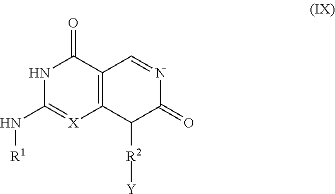

or a pharmaceutically acceptable salt, wherein:
X is CH or nitrogen;
R² is hydrogen or a C₁ to C₂₀ linker group;
Y is absent when R² is hydrogen, or is an amino acid or polypeptide having an amino group covalently bound to an α-carbon of the amino acid and the amino group is covalently bound to the linker group R² when R² is not a hydrogen; and
R¹ is hydrogen or C₁ to C₂₀ aliphatic moiety, such as C₁ to C₂₀ alkyl, straight or branched chain, saturated or unsaturated.

In any aspect or embodiment described herein, the Janus base nanotube comprises a compound of Formula (X):

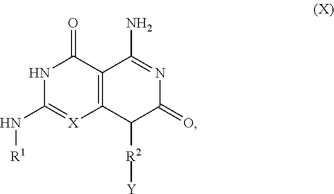

or a pharmaceutically acceptable salt, wherein:
X is CH or nitrogen;
R² is hydrogen or a C₁ to C₂₀ linker group;
Y is absent when R² is hydrogen, or is an amino acid or polypeptide having an amino group covalently bound to an α-carbon of the amino acid and the amino group is covalently bound to the linker group R² when R² is not a hydrogen; and
R¹ is hydrogen or C₁ to C₂₀ aliphatic moiety, such as C₁ to C₂₀ alkyl, straight or branched chain, saturated or unsaturated.

In any aspect or embodiment described herein, the Janus base nanotube comprises a compound of Formula (XI):

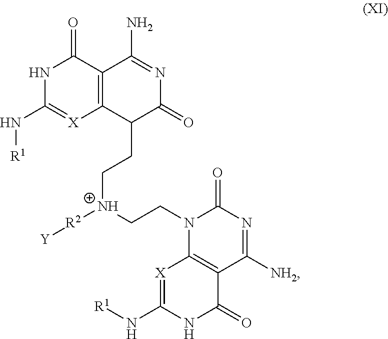

or a pharmaceutically acceptable salt, wherein:
each X is independently CH or nitrogen;
R² is hydrogen or a C₁ to C₂₀ linker group;
Y is absent when R² is hydrogen, or is an amino acid or polypeptide having an amino group covalently bound to an α-carbon of the amino acid and the amino group is covalently bound to the linker group R² when R² is not hydrogen; and
each R¹ is independently hydrogen or a C₁ to C₂₀ aliphatic moiety, such as C₁ to C₂₀ alkyl or straight or branched chain, saturated or unsaturated.

Also disclosed herein are method of coating an electrode (e.g., microelectrode or macroelectrode), the method comprising coating an electrode (e.g., a microelectrode or a macroelectrode) with Janus base nanotubes to create the electrodes disclosed herein. In any aspect or embodiment described herein, coating comprises depositing the Janus base nanotubes on to the surface of the electrode by any appropriate coating method (e.g., pipette deposition, probe dipping, surface lyophilization, laminar flow deposition, microfluidic alignment, and others). In any aspect or embodiment described herein, coating or depositing includes incubating the electrode in a solution comprising Janus base nanotubes. For example, in any aspect or embodiment described herein, coating or depositing includes incubating the electrode in a Janus base nanotubes solution that comprises Janus base nanotubes at a concentration of about 0.001 mg/mL to about 10.0 mg/mL or about 0.1 mg/mL to about 5.0 mg/mL, such as about 1.0 mg/mL

In any aspect or embodiment described herein, the solution of Janus base nanotubes comprises Janus base nanotubes and a liquid. For example, in any aspect or embodiment described herein, the liquid is selected from water, an organic solvent (e.g., methanol, ethanol, dimethyl sulfoxide, dimethylformamide, or a mixture thereof), and a buffer (e.g., phosphate buffer saline, acetate buffer, 2-Morpholinoethanesulfonic acid monohydrate (MES) buffer, tris buffer, water with about 0.0001 μg/mL to about 0.1 mg/mL magnesium ion, or a mixture thereof). For example, in any aspect or embodiment described herein, the solution of Janus base nanotubes comprises Janus base nanotubes and an organic solvent (e.g., methanol, ethanol, dimethyl sulfoxide, dimethylformamide, or a mixture thereof). By way of a further example, in any aspect or embodiment described herein, the solution of Janus base nanotubes comprises Janus base nanotubes and a buffer (e.g., phosphate buffer saline, acetate buffer, 2-Morpholinoethanesulfonic acid monohydrate (MES) buffer, tris buffer, water with about 0.0001 μg/mL to about 0.1 mg/mL magnesium ion, or a mixture thereof). Furthermore, in any aspect or embodiment described herein, the solution of Janus base nanotubes comprises Janus base nanotubes and water.

In any aspect or embodiment described herein, coating comprises drying (e.g., air drying) for a sufficient amount of time to dry the solution.

In any aspect or embodiment described herein, coating and/or depositing is repeated (e.g., repeated to control the thickness of the coating).

In any aspect or embodiment described herein, the method further comprises air-drying, heated-drying, lyophilizing, electrospinning, dipping, or a combination thereof.

Additionally disclosed herein are devices for sensing biological activity, comprising placing the electrode (e.g., microelectrode or macroelectrode) or apparatus of the present disclosure proximate to biological tissue or muscle and monitoring a signal on the electrode or electrically stimulating biological tissue or muscle by way of a wire connected to the electrode, wherein the signal is indicative of the biological activity (e.g., electrical activity or electrical brain activity) of the tissue. Also disclosed herein are methods for sensing biological activity, comprising placing the electrode (e.g., microelectrode or macroelectrode) of the present disclosure, or the apparatus of the present disclosure, proximate to biological tissue or muscle, and monitoring a signal on the electrode or electrically stimulating biological tissue or muscle by way of a wire connected to the electrode, wherein the signal is indicative of the biological activity (e.g., electrical activity or electrical brain activity) of the tissue. In any aspect or embodiment described herein, the biological tissue is brain tissue, heart, muscle, spine tissue, spinal cord tissue, nerve tissue, eye tissue, ear tissue, bone tissue, bone marrow tissue, joint tissue, liver tissue, kidney tissue, lung tissue, bladder tissue, or intestine tissue.

Further disclosed herein are methods of stimulating a biological tissue, the method comprising placing the electrode (e.g., microelectrode or macroelectrode), apparatus, or device of the present disclosure proximate to biological tissue and applying a current and/or a voltage to the biological tissue with the electrode, apparatus, or device, stimulating the biological tissue. For example, in any aspect or embodiment described herein, applying a current and/or a voltage by way a wire connected to the electrode (e.g., microelectrode or macroelectrode).

Also disclosed herein are methods of promoting cell adhesion, the method comprising placing the electrode (e.g., microelectrode or macroelectrode) of the present disclosure, or the apparatus of the present disclosure, proximate to biological tissue, and applying a current and/or a voltage to the biological tissue with the electrode.

Additionally disclosure herein are methods of promoting growth, functions, and/or neurogenesis of a neuronal cell(s) and tissue(s), the method comprising placing the electrode (e.g., microelectrode or macroelectrode) of the present disclosure, or the apparatus of the present disclosure, proximate to biological tissue; and applying a current and/or a voltage to the biological tissue with the electrode.

Further disclosed herein are methods of inhibiting inflammation, the method comprising placing the electrode (e.g., microelectrode or macroelectrode) of the present disclosure, or the apparatus of the present disclosure, proximate to biological tissue; and applying a current and/or a voltage to the biological tissue with the electrode.

The above-described and other features will be appreciated and understood by those skilled in the art from the following detailed description, drawings, and appended claims.

### BRIEF DESCRIPTION OF THE DRAWINGS

The accompanying drawings, which are incorporated into and form a part of the specification, illustrate several embodiments of the present invention and, together with the description, serve to explain the principles of the inventions of the present disclosure. The drawings are only for the purpose of illustrating an embodiment of the invention and are not to be construed as limiting the inventions of the present disclosure. Further objects, features and advantages of the inventions of the present disclosure will become apparent from the following detailed description taken in conjunction with the accompanying figures showing illustrative embodiments of the invention of the present disclosure

FIGS. 1A, 1B, and 1C show the basic structure of Janus Base Nanotubes (JBNTs) from multiple views. FIG. 1A shows a DNA base pair with a lysine side chain that can self-assemble into a helical rosette (FIG. 1B), which is base stacked (e.g., π-π stacking) into the JBNT (FIG. 1C).

FIG. 2 shows the multisite microelectrode probe used during electrochemical measurements, animal study, and nanocoating experiments.

FIGS. 3A, 3B, and 3C show scanning electron microscopy images of an exemplary microelectrode after coating showing bundle formatting near the edges (FIG. 3A), matrix formation near the center (FIG. 3B), and the individual nanotubes in the square of FIG. 3B deposited on the surface of the microelectrode (FIG. 3C).

FIGS. 4A and 4B show a graphical representation of independence values (FIG. 4A) and CV plot values (FIG. 4B) before and after JBNT coating.

FIG. 5 shows morphology characterization of SH-SY5Y cells settled on the JBNT substrate.

FIGS. 6A, 6B, 6C, and 6D show the DNA-mimicking chemical structure of Janus Base Nanotubes (JBNTs). FIG. 6A shows Janus Base Nanotubes (JBNTs) self-assembled into long (>100 nm) tubes via the base-stacking effect. FIG. 6B shows a superior view of G-C pair orientation stabilized through hydrogen bonding. FIG. 6C shows the stacked rosette conformation expanded to demonstrate electron cloud formation and charge distribution between rosettes in assembled conformation. FIG. 6D shows electron transport through axis of JBNT assisted by π-π stacking.

FIG. 6E shows a scanning electron microscopy (SEM) image of a silicon probe with microelectrode sites along the two shanks.

FIGS. 6F and 6G show atomic force microscopy (AFM) images of multiple electrode areas (arrows in FIG. 6E) characterizing height and amplitude of JBNT substrate.

FIGS. 7A, 7B, and 7C show the electrochemical properties of gold and iridium microelectrodes in phosphate-buffered saline solution before and after JBNT coating. FIG. 7A shows the impedance magnitudes were slightly greater and phase angles were more capacitive after coating (top). FIG. 7A shows charge storage capacities were similar, with smaller hydrogen reduction after coating (middle). FIG. 7A shows voltage transient in response to a constant-current pulse was reduced after coating, which is desired (bottom).

FIG. 7B shows the average firing rate, average spike amplitude, and signal-to-noise ratio for iridium microelectrodes (top row) and gold microelectrodes (bottom row) demonstrated similar or slightly improved capability to detect spike waveforms before and after JBNT coating. FIG. 7C shows the sorted units and high-pass filtered waveforms for iridium (top) and gold (bottom) microelectrodes showed qualitatively similar capability to detect spike activity before and after JBNT coating. *P<0.05, **P<0.01, and ***P<0.001 compared to control.

FIGS. 8A, 8B, and 8C show a comparison between JBNT coated probes versus their non-coated control. FIG. 8A show fluorescent microscope (orange) and confocal scan (blue & red) capturing electrode sites after 6-day differentiation of SH-SY5Y cells. Scale bars 100 μm. FIG. 8B shows average DAPI count for iridium and gold electrode probes following 6-day differentiation culture with 50,000 cells/well. FIG. 8C show average DAPI count for iridium and gold electrode probes following a short-term 12-hour incubation period with 100,000 cells/well.

FIG. 8D shows a CCK-8 cytotoxicity assay of JBNT, single-wall carbon nanotubes (SWCNTs), and Polyprryole (PPy) at varying concentrations for 24 hours. Normalized to Control. *P<0.05, **P<0.01, and ***P<0.001 compared to control.

FIGS. 9A, 9B, 9C, and 9D show microscopy images of cell-to-electrode interactions. FIG. 9A shows qualitative interactions of cell-to-substrate behavior of SH-SY5Y cells to negative control gold electrode with no surface modifications. Scale bars 10 μm (top), 2 μm (middle), and 500 nm (bottom). FIG. 9B shows cell-to-substrate interactions between SH-SY5Y cells and JBNT coated Au electrodes sites after 6-day differentiation culture. Scale bars 10 μm (top), 1 μm (middle), and 500 nm (bottom). FIGS. 9C and 9H show colorized cell dispersion on negative control and JBNT coated electrodes respectively. Scale bars 2 μm and 1 μm respectively.

FIGS. 10A and 10B show further microscopy images of cell-to-electrode interactions. FIG. 10A shows JBNT coated iridium electrode at cell-to-electrode interface. FIG. 10B shows AFM scan of amplitude overlaid onto height of cell-to-electrode interface. Scale bars 2 μm and 200 nm respectively.

FIG. 11 shows JBNT coated electrode site following 14-day differentiation culture. High density areas of JBNT remain and indicate integrated anchorage sites.

FIGS. 12A, 12B, 12C, 12D, and 12E show immunological staining of cells and subsequent analysis in cell maturation and growth between experimental groups. FIG. 12A shows full shank assembly of JBNT coated and control probes triple stained for NF-200 (red), NeuN (green), and DAPI (blue). FIG. 12B shows NF-200 and NeuN strain of most dense electrode sites in each experiment group. FIGS. 12C, 12D, and 12E show the average cell count, neuronal nuclei corrected total cell fluorescence and neurofilament growth respectively. Scale bars 100 μm. *P<0.05, **P<0.01, and ***P<0.001 compared to control.

FIG. 13A shows AFM scans of gold electrode sites before and after coating with respective roughness values calculated.

FIG. 13B also shows β1 integrin polyclonal antibody stain on coated silicon dioxide substrates. SH-SY5Y cells were fixed, permeabilized, and stained after 8-hour adhesion period to determine acute timeframe focal adhesion behavior. β1 Integrin expression comparable to collagen substrate while both demonstrate superior scaffolding properties in contrast to negative control. Additional CTCF analysis of β1 Integrin expression demonstrating statistical significance.

FIG. 14 shows the gene ontology results produced after analyzing differentially expressed genes identified during single cell RNA sequencing. Gene ontologies, grouped by cellular components, molecular function, and biological processes are ranked by count and statistical significance. Many of the relevant gene ontology groups are related to focal adhesions, binding affinity, and subsequent neural differentiation.

FIGS. 15A, 5B, 15C, and 15D show the individual genes differentially upregulated or downregulated as a result of binding with the Janus base nanocoating. Heat maps show the upregulation of neurogenic and differentiation activity.

FIGS. 15E, 15F, and 15G show the hypothesized pathways activated during the binding interactions with the Janus base nanocoating. Differential expression analysis suggests heavy involvement from the focal adhesion complex (FAC), which activates downstream signaling pathways involved in neurogenesis and differentiation.

FIG. 16 shows electrochemical properties of the electrode without (middle) and with (bottom) the Janus coating in the rat motor cortex in vivo. Voltage transients in response to constant-current pulses were compatible after coating, which is desired for stimulation applications.

### DETAILED DESCRIPTION

Disclosed herein are electrically conductive coatings based on DNA-inspired Janus base nanotubes (JBNTs) for electrode and insulation areas on electrodes (e.g., microelectrodes or macroelectrodes). The disclosed JBNT coatings are superior to current coating materials. Indeed, they have better biocompatibility and lower cytotoxicity than conductive polymers and carbon nanotubes, and more robust electrical conductivity than bioactive hydrogels. The disclosed JBNT coatings can greatly increase the functions and lifetime of electrodes (e.g., microelectrodes or macroelectrodes) that utilized in human tissues for improved BMI and other applications. Further, the disclosed JBNT coating can release drugs upon chemical or electrical triggers to achieve additional functions such as reducing inflammation, conducting treatment, diagnose diseases, etc.

### Definitions

Throughout the present specification and the accompanying claims the words “comprise,” “include,” and “have” and variations thereof (such as “comprises,” “comprising,” “includes,” “including,” “has,” and “having”) are to be interpreted inclusively or open-ended (that is, to mean including but not limited to). That is, these words are intended to convey the possible inclusion of other elements or integers not specifically recited, where the context allows. No language in the specification should be construed as indicating any non-claimed element essential to the practice of the inventions of the present disclosure. Only the transitional phrases “consisting of” and “consisting essentially of” shall be closed or semi-closed transitional phrases, respectively, as set forth in the United States Patent Office Manual of Patent Examining Procedures, Section 2111.03.

The terms “a,” “an,” and “the” and similar referents used in the context of describing inventions of the present disclosure (especially in the context of the following claims) are to be construed to cover both the singular and the plural (for example, “one or more of” or “at least one”) of the grammatical object of the articles, unless otherwise indicated herein or clearly contradicted by context. By way of example, “an element” means one element or more than one element, unless otherwise indicated.

Recitation of ranges of values herein are merely intended to serve as a shorthand method of referring individually to each separate value falling within the range, unless otherwise indicated herein, each individual value is incorporated into the specification as if it were individually recited herein. Ranges may be expressed herein as from “about” (or “approximately”) one particular value, and/or to “about” (or “approximately”) another particular value. When such a range is expressed, another embodiment includes from the one particular value and/or to the other particular value. Similarly, when values are expressed as approximations, by use of the antecedent “about” or “approximately” it will be understood that the particular value forms another embodiment. It will be further understood that the endpoints of each of the ranges are disclosed both in relation to the other endpoint, and independently of the other endpoint.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc. The use of any and all examples, or exemplary language (e.g., “such as”) provided herein is intended merely to better illuminate the invention and does not pose a limitation on the scope of the invention otherwise claimed.

Units, prefixes, and symbols are denoted in their Systeme International de Unites (SI) accepted form.

Unless otherwise indicated, nucleic acids are written left to right in 5′ to 3′ orientation, and amino acid sequences are written left to right in amino to carboxyl orientation.

Groupings of alternative elements or embodiments of the inventions disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified, thus fulfilling the written description of all Markush groups used in the appended claims.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

Illustrations are for the purpose of describing a preferred embodiment of the inventions of the present disclosure and are not intended to limit the inventions thereto.

As used herein, the term “about” or “approximately” is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated/specified value. For example, the phrase “about 200” includes plus or minus 10% of 200, or from 180 to 220, unless clearly contradicted by context.

As used herein, the term “administering” means the actual physical introduction of a composition into or onto (as appropriate) a host or cell. Any and all methods of introducing the composition into the host or cell are contemplated according to the present disclosure; the method is not dependent on any particular means of introduction and is not to be so construed as such. Means of introduction are well-known to those skilled in the art, and also are exemplified herein.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

### Chemical Definitions

The term “amino acid” refers to a molecule containing both an amino group and a carboxyl group. Exemplary amino acids include, without limitation, both the D- and L-isomers of the naturally-occurring amino acids, as well as non-naturally occurring amino acids prepared by organic synthesis or other metabolic routes. The term amino acid, as used herein, includes without limitation, α-amino acids, natural amino acids, non-natural amino acids, and amino acid analogs.

The term “α-amino acid” refers to a molecule containing both an amino group and a carboxyl group bound to a carbon which is designated the α-carbon.

The term “β-amino acid” refers to a molecule containing both an amino group and a carboxyl group in a β configuration.

The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | 3- | 1- | | Side-chain | |
|---|---|---|---|---|---|
| | Letter | Letter | Side-chain | charge | Hydropathy |
| Amino Acid | Code | Code | Polarity | (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive (10%) | −3.2 |
| | | | | neutral (90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acid” are glycine, alanine, proline, and analogs thereof “Large hydrophobic amino acids” are valine, leucine, isoleucine, phenylalanine, methionine, tryptophan, and analogs thereof “Polar amino acids” are serine, threonine, asparagine, glutamine, cysteine, tyrosine, and analogs thereof “Charged amino acids” are lysine, arginine, histidine, aspartate, glutamate, and analogs thereof.

The term “amino acid analog” refers to a molecule which is structurally similar to an amino acid and that can be substituted for an amino acid in the formation of a peptidomimetic macrocycle. Amino acid analogs include, without limitation, β-amino acids, and amino acids where the amino or carboxyl group is substituted by a similarly reactive group (e.g., substitution of the primary amine with a secondary or tertiary amine, or substitution of the carboxyl group with an ester).

The term “non-natural amino acid” refers to an amino acid that is not one of the twenty amino acids commonly found in peptides synthesized in nature, and known by the one letter abbreviations A, R, N, C, D, Q, E, G, H, I, L, K, M, F, P, S, T, W, Y and V. Non-natural amino acids or amino acid analogs include, without limitation, structures according to the following:

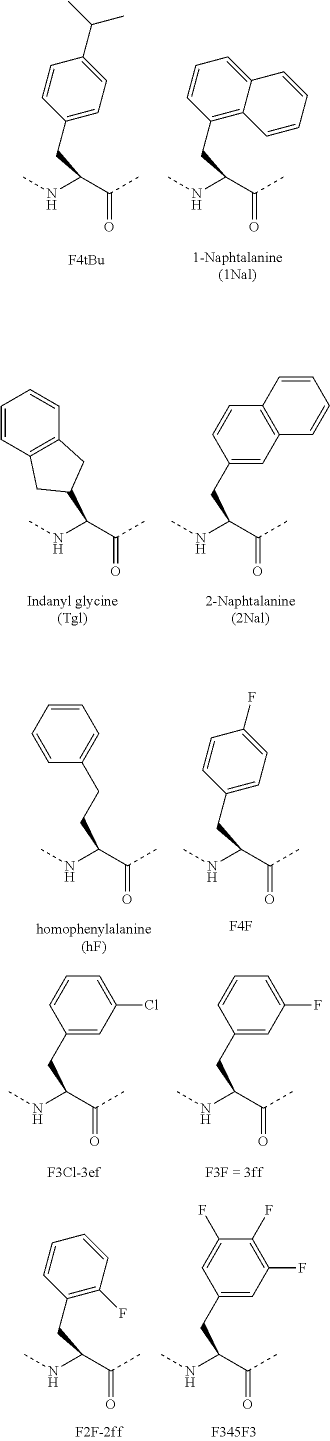

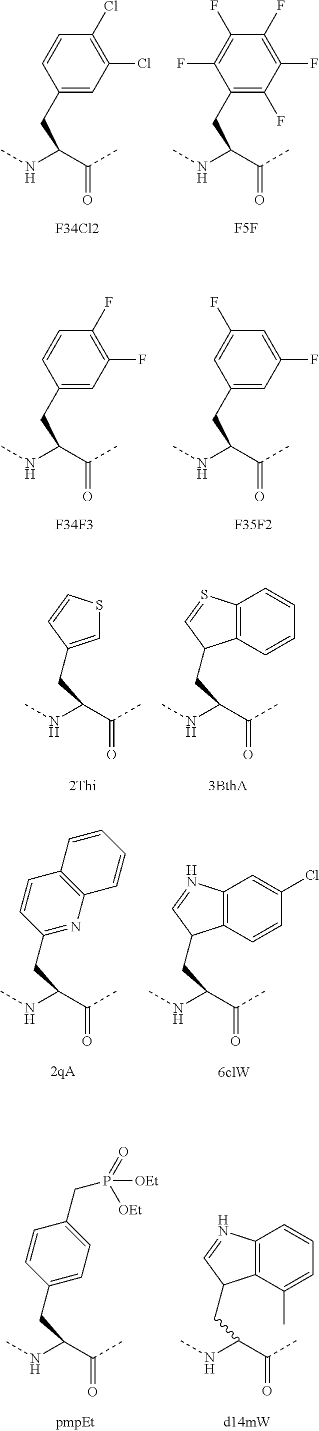

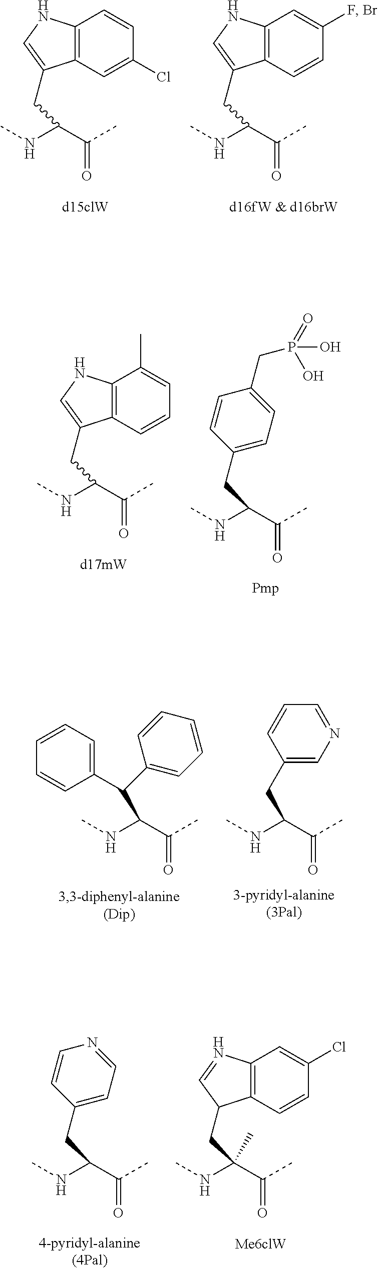

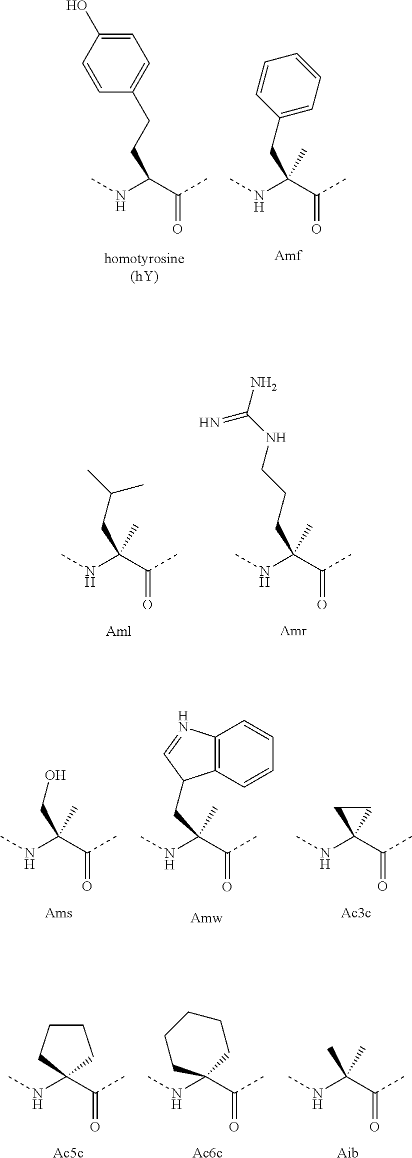

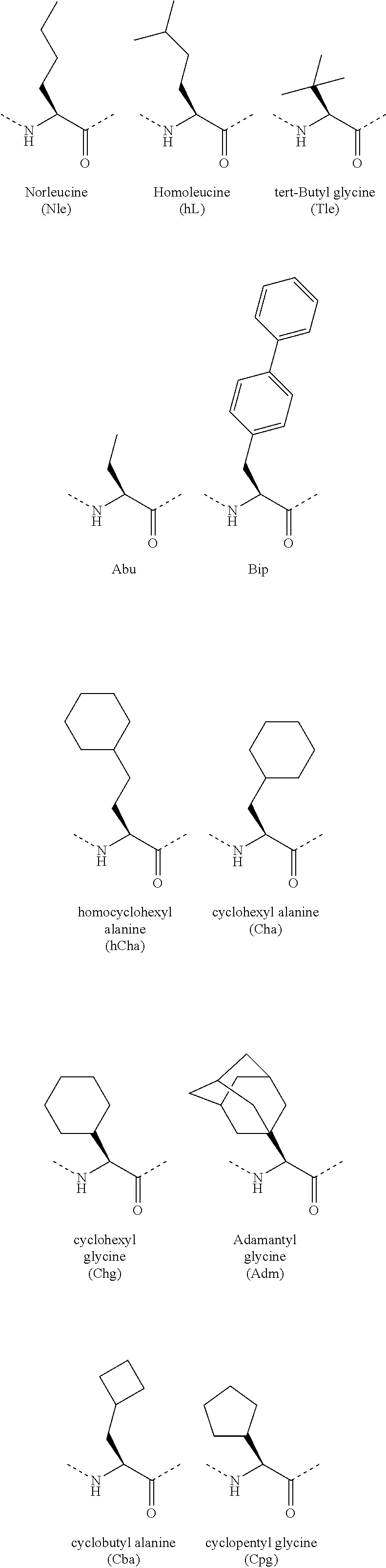

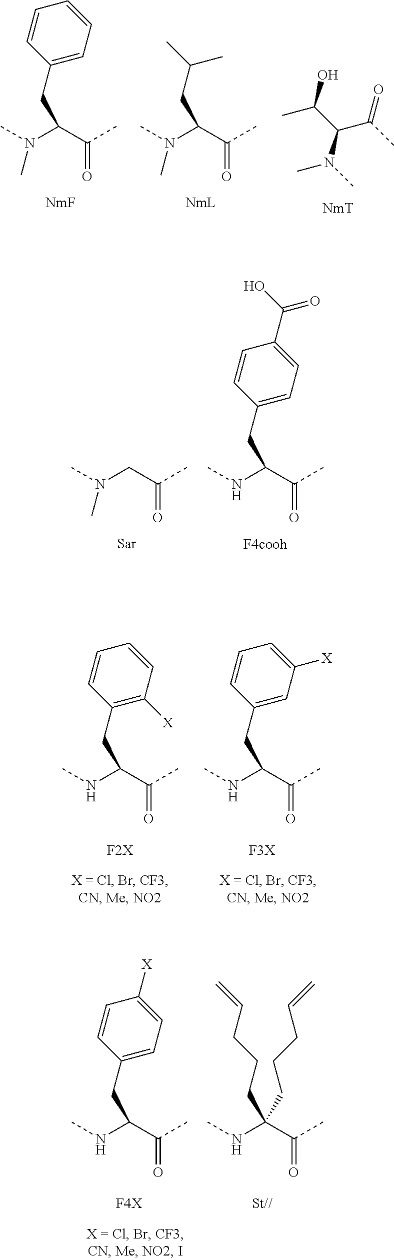

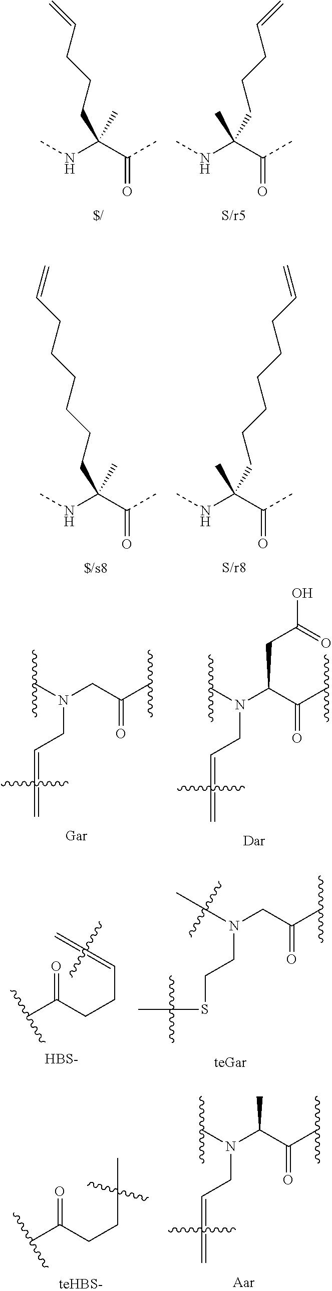

Amino acid analogs include j-amino acid analogs. Examples of β-amino acid analogs include, but are not limited to, the following: cyclic β-amino acid analogs; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl)-butyric acid; (S)-3-amino-4-(4-cyanophenyl)-butyric acid; (S)-3-amino-4-(4-fluorophenyl)-butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; No-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogs include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; 1-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; 1-chloro-L-alanine; 1-cyano-L-alanin; 3-cyclohexyl-D-alanine; 3-cyclohexyl-L-alanine; 3-cyclopenten-1-yl-alanine; 3-cyclopentyl-alanine; 3-cyclopropyl-L-Ala-OH·dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH·dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH·dicyclohexylammonium salt; cyclopentyl-Gly-OH·dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine·dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-0-(dimethylamino)-propionic acid; L-α,β-diaminopropionic acid; L-α-aminobutyric acid; L-α-t-butylglycine; L-β-thienyl)glycine; L-2-amino-3-(dimethylamino)-propionic acid; L-2-aminocaproic acid dicyclohexyl-ammonium salt; L-2-indanylglycine; L-allylglycine·dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogs include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃) OH; Nδ-benzyloxycarbonyl-L-omithine; Nω-nitro-D-arginine; Nω-nitro-L-arginine; α-methyl-omithine; 2,6-diaminoheptanedioic acid; L-omithine; (Nδ-1-(4,4-dimethyl-2,6-dioxo-cyclohex-1-ylidene)ethyl)-D-omithine; (Nδ-1-(4,4-dimethyl-2,6-dioxo-cyclohex-1-ylidene)ethyl)-L-omithine; (Nδ-4-methyltrityl)-D-omithine; (Nδ-4-methyltrityl)-L-omithine; D-omithine; L-omithine; Arg(Me)(Pbf)-OH; Arg(Me)2-OH (asymmetrical); Arg(Me)2-OH (symmetrical); Lys(ivDde)-OH; Lys(Me)2-OH·HCl; Lys(Me3)-OH chloride; Nω-nitro-D-arginine; and Nω-nitro-L-arginine.

Amino acid analogs include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; 7-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-ca-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)-OH; and pyroglutamic acid.

Amino acid analogs include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Amino acid analogs include analogs of phenylalanine and tyrosine. Examples of amino acid analogs of phenylalanine and tyrosine include 3-methyl-phenylalanine, 3-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, β-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogs include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

Amino acid analogs include analogs of serine and threonine. Examples of amino acid analogs of serine and threonine include, but are not limited to, β-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogs include analogs of tryptophan. Examples of amino acid analogs of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In any aspect or embodiment described herein, amino acid analogs are racemic. In any aspect or embodiment described herein, the D isomer of the amino acid analog is used. In some embodiments, the L isomer of the amino acid analog is used. In other embodiments, the amino acid analog comprises chiral centers that are in the R or S configuration. In still other embodiments, the amino group(s) of a β-amino acid analog is substituted with a protecting group, e.g., tert-butyloxycarbonyl (BOC group), 9-fluorenylmethyloxycarbonyl (FMOC), tosyl, and the like. In yet other embodiments, the carboxylic acid functional group of a β-amino acid analog is protected, e.g., as its ester derivative. In some embodiments the salt of the amino acid analog is used.

A “non-essential” amino acid residue is a residue that can be altered from the wild-type sequence of a polypeptide without abolishing or substantially abolishing its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, for example, is replaced with another amino acid residue from the same side chain family. Other examples of acceptable substitutions are substitutions based on isosteric considerations (e.g. norleucine for methionine) or other properties (e.g., 2-thienylalanine for phenylalanine).

The term “polypeptide” refers to a linear organic polymer consisting of a large number of amino-acid residues bonded together in a chain, forming part of (or the whole of) a protein molecule.

The term “α-polypeptide” refers to are polypeptides derived from α-amino acids.

The term “β-polypeptide” refers to are polypeptides derived from β-amino acids.

The term “aliphatic” or “aliphatic group” refers to a hydrocarbon moiety that may be straight-chain (i.e., unbranched), branched, or cyclic (including fused, bridging, and spiro-fused polycyclic) and may be completely saturated or may contain one or more units of unsaturation. Suitable aliphatic groups include, but are not limited to, linear or branched, alkyl, alkenyl, and alkynyl groups, and hybrids thereof. As used herein the terms “aliphatic” or “aliphatic group”, also encompass partially substituted analogs of these moieties where at least one of the hydrogen atoms of the aliphatic group is replaced by an atom that is not carbon or hydrogen.

The term “linker” refers to a chemical group that connects one or more other chemical groups via at least one covalent bond.

While the invention has been described with reference to an exemplary embodiments, it will be understood by those skilled in the art that various changes may be made and equivalents may be substituted for elements thereof without departing from the scope of the invention. In addition, many modifications may be made to adapt a particular situation or material to the teachings of the inventions of the present disclosure without departing from the essential scope thereof. Therefore, it is intended that the inventions of the present disclosure not to be limited to the particular embodiments disclosed as the best mode contemplated for carrying out the inventions of the present disclosure, but that the inventions of the present disclosure will include all embodiments falling within the scope of the appended claims. Any combination of the described elements in all possible variations thereof is encompassed by the inventions of the present disclosure unless otherwise indicated herein or otherwise clearly contradicted by context.

### Electrodes

An aspect of the present disclosure relates to an electrode (e.g., a microelectrode or macroelectrode) comprising a nanomaterial coating disposed on the electrode, wherein the coating comprises a Janus base nanotube. In any aspect or embodiment described herein, the electrode further comprises insulation areas disposed on the electrode.

In any aspect or embodiment described herein, the electrode is a microelectrode. For example, in any aspect or embodiment described herein, the electrode is a microelectrode with a geometric surface area of less than about 10000 μm², less than about 9000 μm², less than about 8000 μm², less than about 7000 μm², less than about 6000 μm², less than about 5000 μm², less than about 4000 μm², less than about 3000 μm², less than about 2000 μm², or less than about 1000 μm².

In any aspect or embodiment described herein, the electrode is a macroelectrode. For example, in any aspect or embodiment described herein, the electrode is a macroelectrode with a geometric surface area of at least about 10,000 μm², at least about 11,000 μm², at least about 12,000 μm², at least about 13,000 μm², at least about 14,000 μm², at least about 15,000 μm², less than about 4000 μm², at least about 16,000 μm², at least about 17,000 μm², at least about 18,000 μm², at least about 19,000 μm², or at least about 20,000 μm².

In any aspect or embodiment described herein, the nanomaterial coating further comprises one or more therapeutic molecules. For example, in any aspect or embodiment described herein, the one or more therapeutic molecules are selected from Transforming Growth Factor-β (TGFβ), Vascular Endothelial Growth Factor (VEGF), an Insulin Like Growth Factor (IGF), an Epidermal Growth Factor (EGF), a Platelet-Derived Growth Factor (PDGF), a Bone Morphogenetic Protein (BMP), a Fibroblast Growth Factor (FGF), Glial Cell Derived Neurotrophic Factor (GDNF), Hepatocyte Growth Factor (HGF), Placental Growth Factor (PGF), Nerve Growth Factor (NGF), Tumor Necrosis Factor-α (TNF-α), Stromal Cell-Derived Factor 1 9SDF-1), dexamethasone, resveratrol a small interfering ribonucleic acid (siRNA), a micro ribonucleic acid (miRNA), a growth factor, a small-molecule drug, the like, and mixtures thereof.

In any aspect or embodiment described herein, the nanomaterial coating is a self-assembled nanomaterial coating.

In any aspect or embodiment described herein, the nanomaterial coating is a single compartment nanomaterial coating. As used herein, a single compartment nanomaterial includes a single population of self-assembled nanomaterials, that is, a single type of JBNT, which can include one or more therapeutic molecules.

In any aspect or embodiment described herein, the nanomaterial coating is a multiple compartment nanomaterial coating. As used herein, a multiple compartment nanomaterial includes two or more populations of self-assembled nanomaterials that form a multi-compartmental structure, such as a layer-by-layer assembly (e.g., through electrostatic layer-by-layer assembly), which can include one or more therapeutic molecules. For example, opposite electrostatic charges on the first and second populations of JBNTs can drive assembly of the multiple compartment nanomaterial. By assembling the first and second populations in order, the one population will form the interior compartment and the second population will form the exterior compartment.

Another aspect of the present disclosure relates to an apparatus or device comprising the electrode (e.g., microelectrode or macroelectrode) of the present disclosure. For example, an aspect of the present disclosure relates to an apparatus or device comprising an electrode (e.g., a microelectrode or macroelectrode), wherein the electrode comprises a nanomaterial coating disposed on the electrode, wherein the coating comprises a Janus base nanotube.

In any aspect or embodiment described herein, the electrode (e.g., microelectrode or macroelectrode) coatings of the present disclosure are electrically conductive coating based on DNA-inspired Janus base nanotubes (JBNTs). In any aspect or embodiment described herein, the JBNTS comprise a compound selected from a compound of Formula (I), (II), (III), (IV), (V), (VI), (VII), (VIII), (IX), (X), (XI), and combinations thereof.

Also disclosed herein are apparatuses or devices that comprise an electrode(s) (e.g., microelectrode(s) or macroelectrode(s)) of the present disclosure. For example, in any aspect or embodiment described herein, an apparatus comprises an electrode (e.g., a microelectrode or macroelectrode) that comprises a nanomaterial coating disposed on the electrode, wherein the coating comprises a Janus base nanotube. In any aspect or embodiment described herein, the electrode further comprises insulation areas disposed on the electrode.

### Devices

The presently disclosed electrodes (e.g., microelectrodes or macroelectrodes) have a wide array of applications, including, but not limited to biological applications. For example, in some embodiments, disclosed herein, are devices for sensing biological activity comprising placing the electrode (e.g., microelectrode or macroelectrode) of the present disclosure proximate to biological tissue or muscle; monitoring a signal on the electrode or electrically stimulating biological tissue or muscle by way of a wire connected to the electrode; and wherein the signal is indicative of the biological activity (e.g., electrical activity or electrical brain activity) of the tissue.

### Methods of Preparing

The presently disclosed electrodes (e.g., microelectrodes and macroelectrodes) can be prepared by any appropriate method of coating an electrode with Janus base nanotubes. For example, an aspect of the present disclosure relates to a method of coating an electrode (e.g., a microelectrode or macroelectrode), the method comprising coating an electrode with Janus base nanotubes to create the electrodes disclosed herein. In any aspect or embodiment described herein, coating comprises depositing the Janus base nanotubes on to the surface of the electrode by any appropriate coating method (e.g., pipette deposition, probe dipping, surface lyophilization, laminar flow deposition, microfluidic alignment, and others). In any aspect or embodiment described herein, coating or depositing includes incubating the electrode in a solution comprising Janus base nanotubes. For example, in any aspect or embodiment described herein, coating or depositing includes incubating the electrode in a Janus base nanotubes solution that comprises Janus base nanotubes at a concentration of about 0.001 mg/mL to about 10.0 mg/mL or about 0.1 mg/mL to about 5.0 mg/mL, such as about 1 mg/mL. For example, in any aspect or embodiment described herein, coating or depositing includes incubating the microelectrode in a Janus base nanotubes solution that comprises Janus base nanotubes at a concentration of about 0.001 mg/mL to about 10.0 mg/mL, about 0.01 mg/mL to about 10.0 mg/mL, about 0.1 mg/mL to about 10.0 mg/mL, about 1.0 mg/mL to about 10.0 mg/mL, about 2.0 mg/mL to about 10.0 mg/mL, about 3.0 mg/mL to about 10.0 mg/mL, about 4.0 mg/mL to about 10.0 mg/mL, about 5.0 mg/mL to about 10.0 mg/mL, about 6.0 mg/mL to about 10.0 mg/mL, about 7.0 mg/mL to about 10.0 mg/mL, about 0.001 mg/mL to about 9.0 mg/mL, about 0.01 mg/mL to about 9.0 mg/mL, about 0.1 mg/mL to about 9.0 mg/mL, about 1.0 mg/mL to about 9.0 mg/mL, about 2.0 mg/mL to about 9.0 mg/mL, about 3.0 mg/mL to about 9.0 mg/mL, about 4.0 mg/mL to about 9.0 mg/mL, about 5.0 mg/mL to about 9.0 mg/mL, about 6.0 mg/mL to about 9.0 mg/mL, about 0.001 mg/mL to about 8.0 mg/mL, about 0.01 mg/mL to about 8.0 mg/mL, about 0.1 mg/mL to about 8.0 mg/mL, about 1.0 mg/mL to about 8.0 mg/mL, about 2.0 mg/mL to about 8.0 mg/mL, about 3.0 mg/mL to about 8.0 mg/mL, about 4.0 mg/mL to about 8.0 mg/mL, about 5.0 mg/mL to about 8.0 mg/mL, about 0.001 mg/mL to about 7.0 mg/mL, about 0.01 mg/mL to about 7.0 mg/mL, about 0.1 mg/mL to about 7.0 mg/mL, about 1.0 mg/mL to about 7.0 mg/mL, about 2.0 mg/mL to about 7.0 mg/mL, about 3.0 mg/mL to about 7.0 mg/mL, about 4.0 mg/mL to about 7.0 mg/mL, about 0.001 mg/mL to about 6.0 mg/mL, about 0.01 mg/mL to about 6.0 mg/mL, about 0.1 mg/mL to about 6.0 mg/mL, about 1.0 mg/mL to about 6.0 mg/mL, about 2.0 mg/mL to about 6.0 mg/mL, about 3.0 mg/mL to about 6.0 mg/mL, about 0.001 mg/mL to about 5.0 mg/mL, about 0.01 mg/mL to about 5.0 mg/mL, about 0.1 mg/mL to about 5.0 mg/mL, about 1.0 mg/mL to about 5.0 mg/mL, about 2.0 mg/mL to about 5.0 mg/mL, about 0.001 mg/mL to about 4.0 mg/mL, about 0.01 mg/mL to about 4.0 mg/mL, about 0.1 mg/mL to about 4.0 mg/mL, about 1.0 mg/mL to about 4.0 mg/mL, about 0.001 mg/mL to about 3.0 mg/mL, about 0.01 mg/mL to about 3.0 mg/mL, about 0.1 mg/mL to about 3.0 mg/mL, about 0.001 mg/mL to about 2.0 mg/mL, about 0.01 mg/mL to about 2.0 mg/mL, about 0.1 mg/mL to about 2.0 mg/mL, about 0.001 mg/mL to about 1.0 mg/mL, or about 0.01 mg/mL to about 1.0 mg/mL.

In any aspect or embodiment described herein, the solution of Janus base nanotubes comprises Janus base nanotubes and a liquid. For example, in any aspect or embodiment described herein, the liquid is selected from water, an organic solvent (e.g., methanol, ethanol, dimethyl sulfoxide, dimethylformamide, or a mixture thereof), and a buffer (e.g., phosphate buffer saline, acetate buffer, 2-Morpholinoethanesulfonic acid monohydrate (MES) buffer, tris buffer, water with about 0.0001 μg/mL to about 0.1 mg/mL magnesium ion, or a mixture thereof). For example, in any aspect or embodiment described herein, the solution of Janus base nanotubes comprises Janus base nanotubes and water. By way of a further example, in any aspect or embodiment described herein, the solution of Janus base nanotubes comprises Janus base nanotubes and an organic solvent (e.g., methanol, ethanol, dimethyl sulfoxide, dimethylformamide, or a mixture thereof). In a further example, in any aspect or embodiment described herein, the solution of Janus base nanotubes comprises Janus base nanotubes and a buffer (e.g., phosphate buffer saline, acetate buffer, 2-Morpholinoethanesulfonic acid monohydrate (MES) buffer, tris buffer, water with about 0.0001 μg/mL to about 0.1 mg/mL magnesium ion, or a mixture thereof).

In any aspect or embodiment described herein, coating comprises drying (e.g., air drying) for a sufficient amount of time to dry the solution. In any aspect or embodiment described herein, the method further comprises air-drying, heated-drying, lyophilizing, electrospinning, dipping, or a combination thereof. In any aspect or embodiment described herein, coating and/or depositing is repeated (e.g., repeated to control the thickness of the coating).

### Methods of Use

In any aspect or embodiment described herein, the electrodes (e.g., microelectrodes or macroelectrodes), apparatus, and devices of the present disclosure can be used in various applications, including, without limitation, medical applications. Specifically, but not exclusively, the electrodes (e.g., microelectrodes or macroelectrodes), apparatus, and devices of the present disclosure can be used for deep brain stimulation, neuromonitoring, spinal stimulation, peripheral nerve stimulation, cardiac monitoring, cardiac rhythm management, ablation, mapping, and the like.

A further aspect of the present disclosure relates to a method for sensing biological activity, the method comprising placing the electrodes (e.g., microelectrodes or macroelectrodes) or apparatus of the present disclosure proximate to biological tissue or muscle and monitoring a signal on the electrode by way of a wire connected to the electrode, wherein the signal is indicative of the biological activity (e.g., electrical activity or electrical brain activity) of the tissue. In any aspect or embodiment described herein, the biological tissue is brain tissue, heart, muscle, spine tissue, spinal cord tissue, nerve tissue, eye tissue, ear tissue, bone tissue, bone marrow tissue, joint tissue, liver tissue, kidney tissue, lung tissue, bladder tissue, or intestine tissue.

Another aspect of the present disclosure relates to a method of stimulating a biological tissue, the method comprising placing the electrode (e.g., microelectrode or macroelectrode), apparatus, or device of the present disclosure proximate to biological tissue and applying a current and/or a voltage to the biological tissue with the electrode, apparatus, or device, stimulating the biological tissue. For example, in any aspect or embodiment described herein, applying a current and/or a voltage by way a wire connected to the electrode. In any aspect or embodiment described herein, the biological tissue is brain tissue, heart, muscle, spine tissue, spinal cord tissue, nerve tissue, eye tissue, ear tissue, bone tissue, bone marrow tissue, joint tissue, liver tissue, kidney tissue, lung tissue, bladder tissue, or intestine tissue.

An additional aspect of the present disclosure relates to a method of promoting cell adhesion, the method comprising placing the electrode (e.g., microelectrode or macroelectrode) of the present disclosure, or the apparatus of the present disclosure, proximate to biological tissue, and applying a current and/or a voltage to the biological tissue with the electrode.

Yet a further aspect of the present disclosure relates to a method of promoting growth, functions, and/or neurogenesis of a neuronal cell(s) and tissue(s), the method comprising placing the electrode (e.g., microelectrode or macroelectrode) of the present disclosure, or the apparatus of the present disclosure, proximate to biological tissue; and applying a current and/or a voltage to the biological tissue with the electrode.

An additional aspect of the present disclosure relates to a method of inhibiting inflammation, the method comprising placing the electrode (e.g., microelectrode or macroelectrode) of the present disclosure, or the apparatus of the present disclosure, proximate to biological tissue; and applying a current and/or a voltage to the biological tissue with the electrode.

### Janus Base Nanotubes

The electrode coatings of the present disclosure are electrically conductive coating based on DNA-inspired Janus base nanotubes (JBNTs). In some embodiments, these JBNTS comprise a compound selected from a compound of Formula (I), (II), (III), (IV), (V), (VI), (VII), (VIII), (IX), (X), (XI), and combinations thereof.

In any aspect or embodiment described herein, the Janus base nanotube comprises a compound of Formula (I):

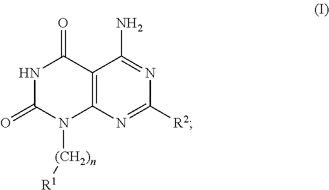

or a pharmaceutically acceptable salt thereof, wherein:
n is 1, 2, 3, 4, 5, or 6;
R¹ is selected from an α-amino acid, a β-amino acid, an α-polypeptide, and a β-polypeptide,
R² is selected from H, CH₃, and NHR^{z}; and
R^{z} is H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (III):

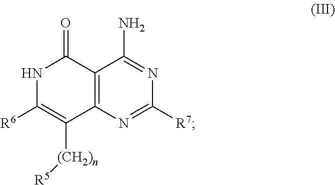

or a pharmaceutically acceptable salt thereof, wherein:
n is 1, 2, 3, 4, 5, or 6;
R⁵ is selected from an α-amino acid, a β-amino acid, an α-polypeptide, and a β-polypeptide,
each of R⁶ and R⁷ is independently selected from H, CH₃, and NHR^{z}; and
each R^{z} is H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (V):

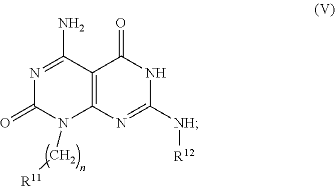

or a pharmaceutically acceptable salt or ester thereof, wherein:
n is 1, 2, 3, 4, 5, or 6;
R¹¹ is selected from an α-amino acid, a β-amino acid, an α-polypeptide, and a β-polypeptide; and
R¹² is H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (VII):

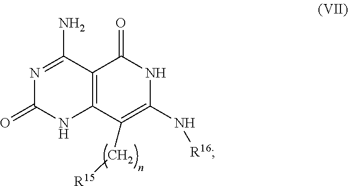

or a pharmaceutically acceptable salt or ester thereof, wherein:
n is 1, 2, 3, 4, 5, or 6;
R¹⁵ is selected from an α-amino acid, a β-amino acid, an α-polypeptide, and a β-polypeptide; and
R¹⁶ is H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (II):

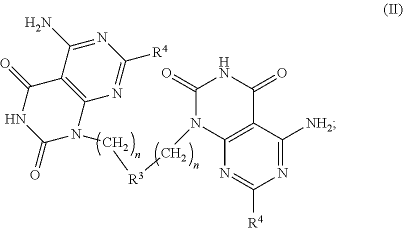

or a pharmaceutically acceptable salt thereof, wherein:
each n is 1, 2, 3, 4, 5, or 6;
R³ is selected from an α-amino acid, a β-amino acid, an α-polypeptide, and a β-polypeptide;
each R⁴ is independently H, CH₃, or NHR^{z}; and
each R^{z} is independently H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (IV):

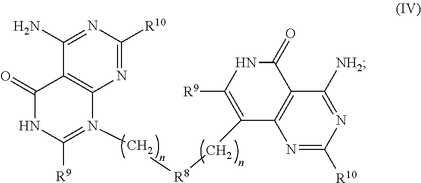

or a pharmaceutically acceptable salt thereof, wherein
each n is 1, 2, 3, 4, 5, or 6;
R⁸ is selected from an α-amino acid, a β-amino acid, an α-polypeptide, and a β-polypeptide;
each R⁹ and R¹⁰ is independently H, CH₃, or NHR^{z}; and
each R^{z} is independently H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (VI):

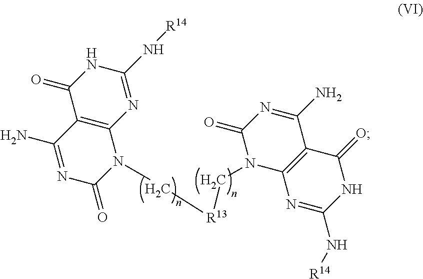

or a pharmaceutically acceptable salt thereof, wherein
each n is 1, 2, 3, 4, 5, or 6;
R¹³ is selected from an α-amino acid, a β-amino acid, an α-polypeptide, and a β-polypeptide; and
each R¹⁴ is independently H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (VIII):

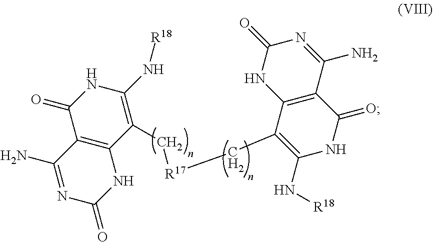

or a pharmaceutically acceptable salt thereof, wherein:
each n is 1, 2, 3, 4, 5, or 6;
R¹⁷ is selected from an α-amino acid, a β-amino acid, an α-polypeptide, and a β-polypeptide; and
each R¹⁸ is independently H or a C₁ to C₂₀ aliphatic group, such as C₁ to C₂₀ alkyl, straight or branched chain, saturated or unsaturated.

In any aspect or embodiment described herein, the Janus base nanotube comprises a compound of Formula (IX):

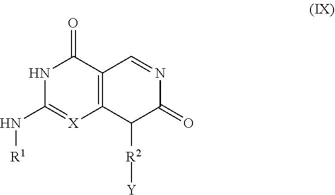

or a pharmaceutically acceptable salt, wherein:
X is CH or nitrogen;
R² is hydrogen or a C₁ to C₂₀ linker group;
Y is absent when R² is hydrogen, or is an amino acid or polypeptide having an amino group covalently bound to an α-carbon of the amino acid and the amino group is covalently bound to the linker group R² when R² is not hydrogen; and
R¹ is hydrogen or C₁ to C₂₀ aliphatic moiety, such as straight or branched chain, saturated or unsaturated C₁ to C₂₀ alkyl.

In any aspect or embodiment described herein, the Janus base nanotube comprises a compound of Formula (X):

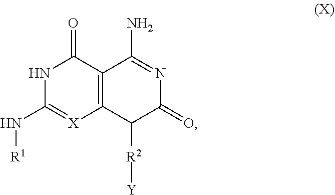

or a pharmaceutically acceptable salt, wherein:
X is CH or nitrogen;
R² is hydrogen or a C₁ to C₂₀ linker group;
Y is absent when R² is hydrogen, or is an amino acid or polypeptide having an amino group covalently bound to an α-carbon of the amino acid and the amino group is covalently bound to the linker group R² when R2 is not hydrogen; and
R¹ is hydrogen or C₁ to C₂₀ aliphatic moiety, such as straight or branched chain, saturated or unsaturated C₁ to C₂₀ alkyl.

In any aspect or embodiment described herein, the Janus base nanotube comprises a compound of Formula (XI),

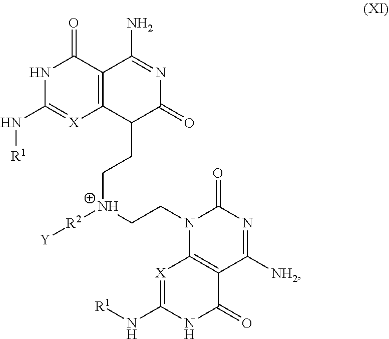

or a pharmaceutically acceptable salt, wherein:
X is CH or nitrogen;
R₂ is hydrogen or a C₁ to C₂₀ linker group;
Y is absent when R² is hydrogen, or is an amino acid or polypeptide having an amino group covalently bound to an α-carbon of the amino acid and the amino group is covalently bound to the linker group R² when R² is not hydrogen; and
R¹ is hydrogen or a C1 to C20 aliphatic moiety, such as straight or branched chain, saturated or unsaturated C1 to C20 alkyl.

### EXEMPLIFICATION

### Example 1: Electrically Conductive Janus Base Nano-Coating for Brain Machine Interface Enhancement

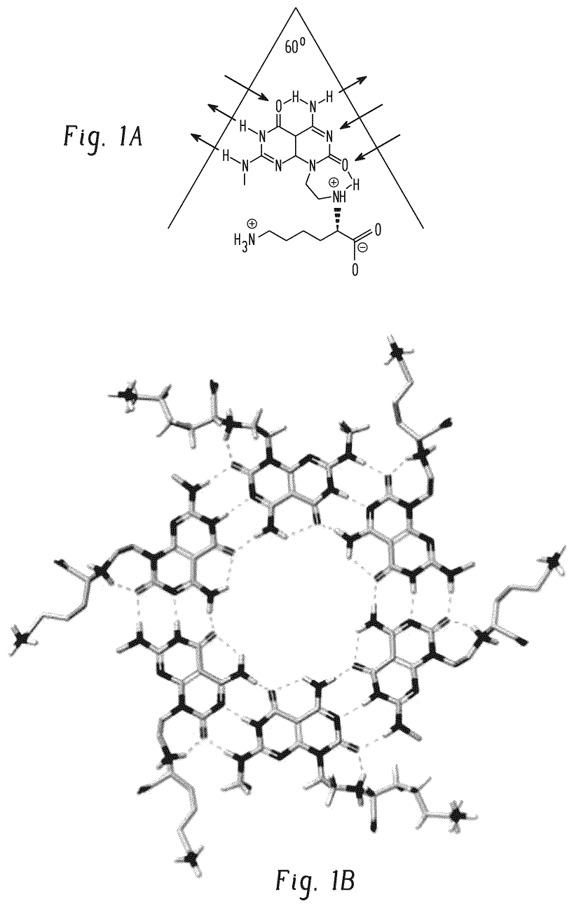

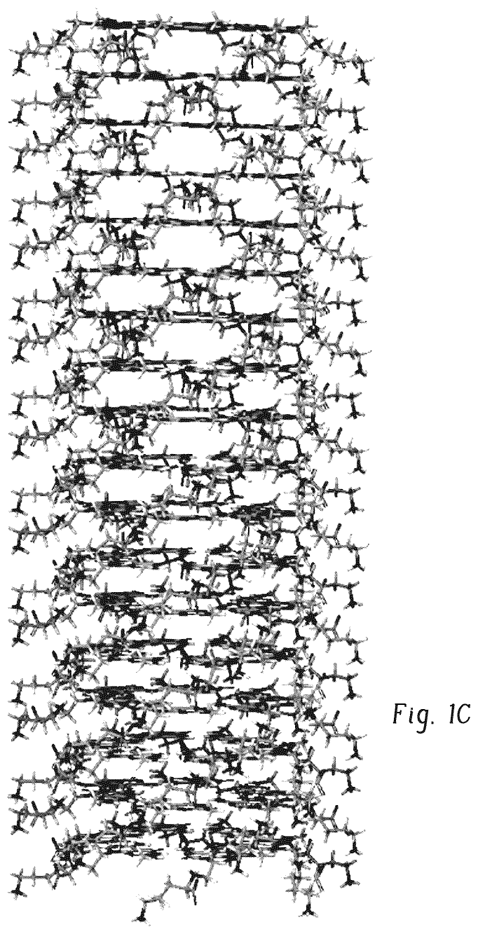

Disclosed in this example is an electrically conductive coating based on DNA-inspired Janus Base Nanotubes (JBNTs). JBNTs are a family of nanotubes self-assembled from G C units mimicking DNA base pairs via hydrogen bonds and the base stacking effect (FIG. 1A-1C). The self-assembled DNA-mimicking structure of JBNTs has demonstrated superior biocompatibility with various types of cells including neurons, which suggests their promising potential as a BMI electrode coating. Furthermore, long distance electron dislocation due to π-π interactions allow JBNTs to conduct electrical signal at the interface between tissue and electrode. Due to hydrogen bonding and base stacking during self-assembly, JBNTs provide excellent biodegradability and present no cytotoxicity compared to carbon nanotubes (CNTs) and other polymers.

### Materials and Methods

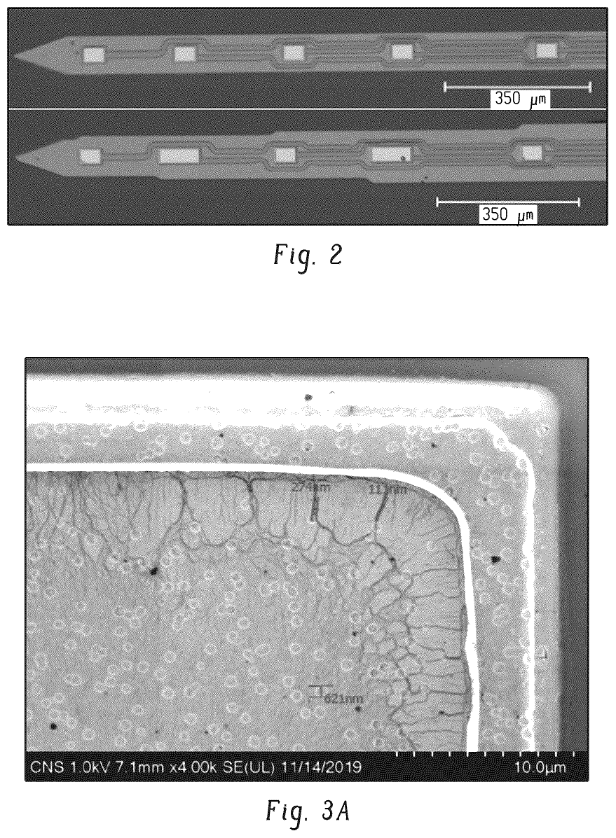

The microelectrode probe (FIG. 2) was broken, and the adjacent probe was used for subsequent testing. The probe was dipped in phosphate-buffered saline (PBS), then submerged in 10 mg/mL JBNT solution for two hours. Probe was then removed from the JBNT solution and allowed to air dry for one hour. Electrochemical measurements were then taken, including impedance and charge injection before and after JBNT nano-coating. A non-survival study was then conducted in order to assess the device's stimulation and recording properties in the rat motor cortex in vivo. SEM images were then taken to characterize and further investigate the coating profile. SH-SY5Y neural cells were then cultured on representative JBNT coated glass wells to demonstrate adhesion through qualitative analysis. Substrates were coated with 100 μL of JBNT (1 mg/mL) and lyophilized overnight. Cells were cultured 4 hours then fixed and stained for imaging. Single cell RNA sequencing (scRNA-seq) was then performed on cells bound to JBNT nano-coating versus control after two weeks of in vitro culture in order to analyze individual transcriptomes for differentially expressed genes.

### Results

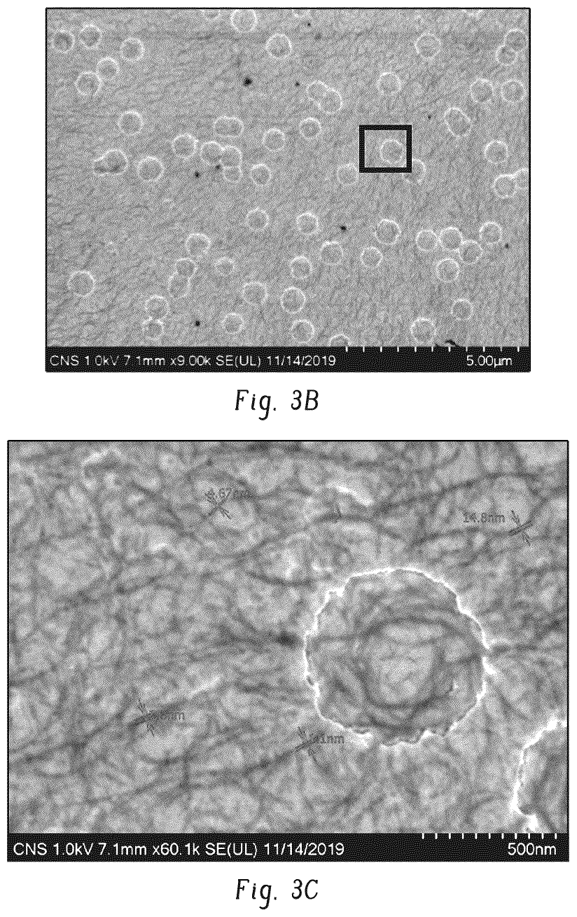

JBNTs displayed nearly homogenous coating on surface of electrodes (FIGS. 3A, 3B, and 3C). Thicker bundles formed closer to the electrode perimeter.

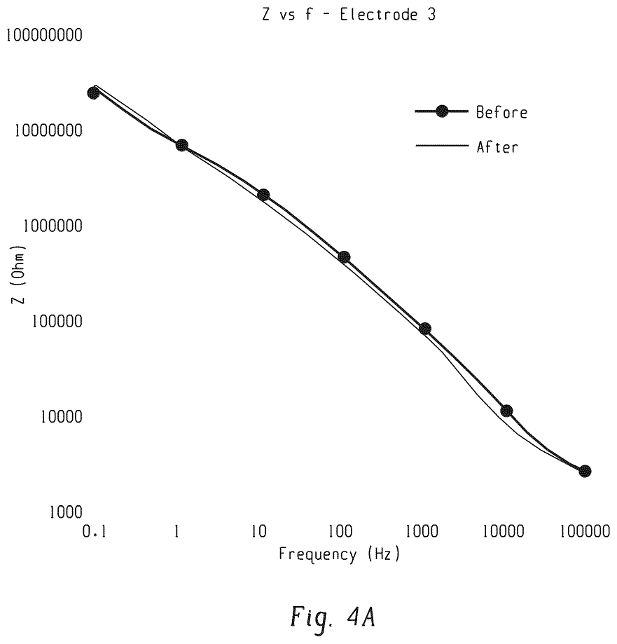

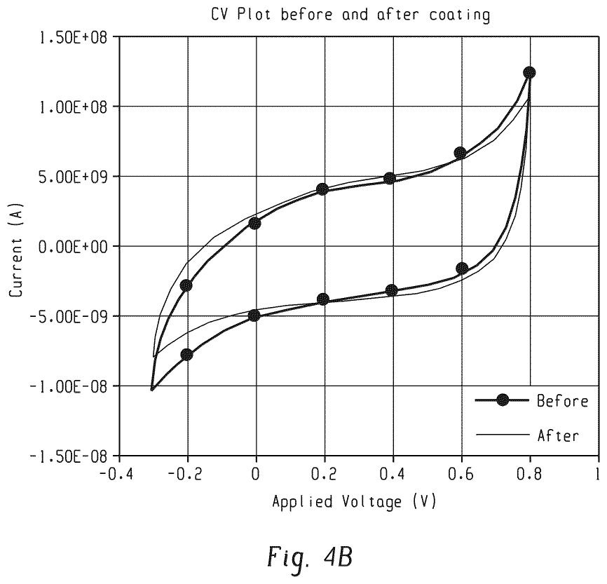

As seen in FIG. 4A, impedance values were largely undisturbed with respect to standard uncoated values. Impedance at 1 kHz before and after coating was 85.6 kΩ and 74 kΩ, respectively.

In FIG. 4B, there was no significant change in size or shape of the current (A)-voltage (V) plot before and after coating. Furthermore, the Charge Storage Capacity (CSC) before and after coating was 4.97 mC/cm² and 4.61 mC/cm², respectively.

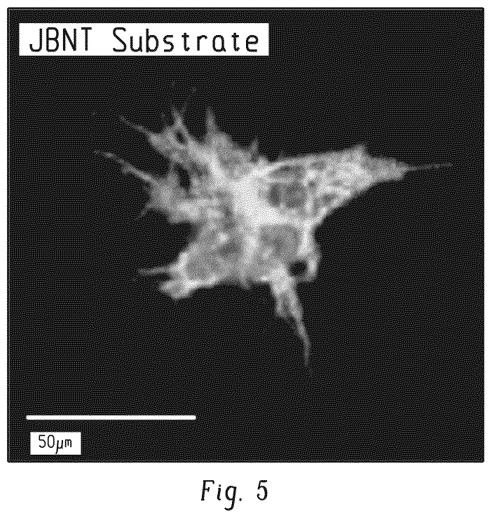

FIG. 5 shows initial cellular adhesion on JBNT substrate and demonstrates that neuron cells had a good cell-to substrate focal adhesions with significant filament protrusions. This result demonstrates the excellent biocompatibility of the JBNTs.

### CONCLUSIONS

These results demonstrate that JBNT has great promise as a novel biomimetic coating for BMI interface enhancement.

### Example 2: Nanomaterial Coating for Electrodes

In Example 2, electrically conductive coating is developed based on DNA-inspired Janus base nanotubes (JBNTs) for electrode and insulation areas on microelectrodes.

### Coating and Characterization

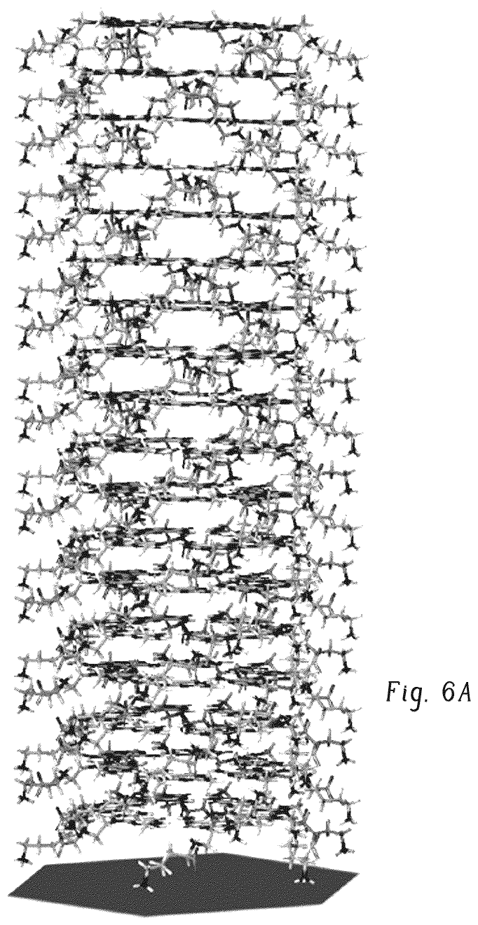

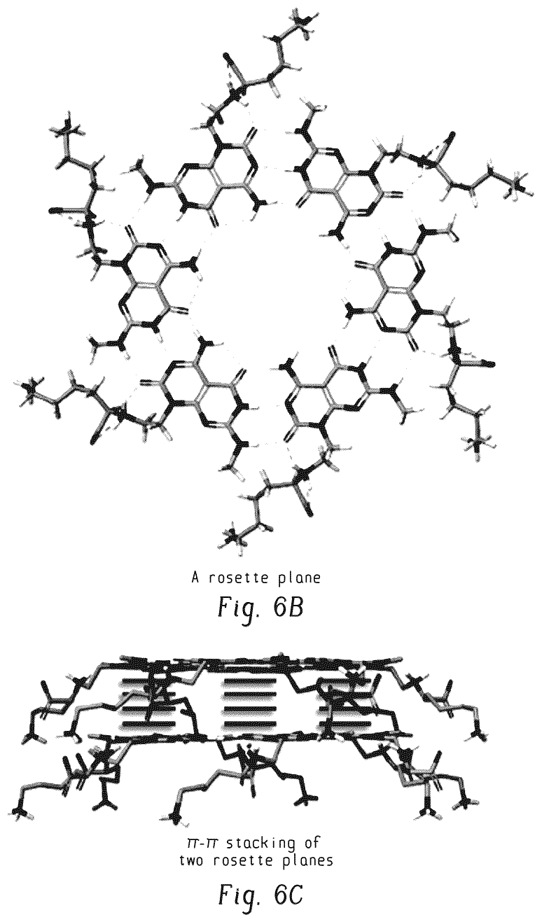

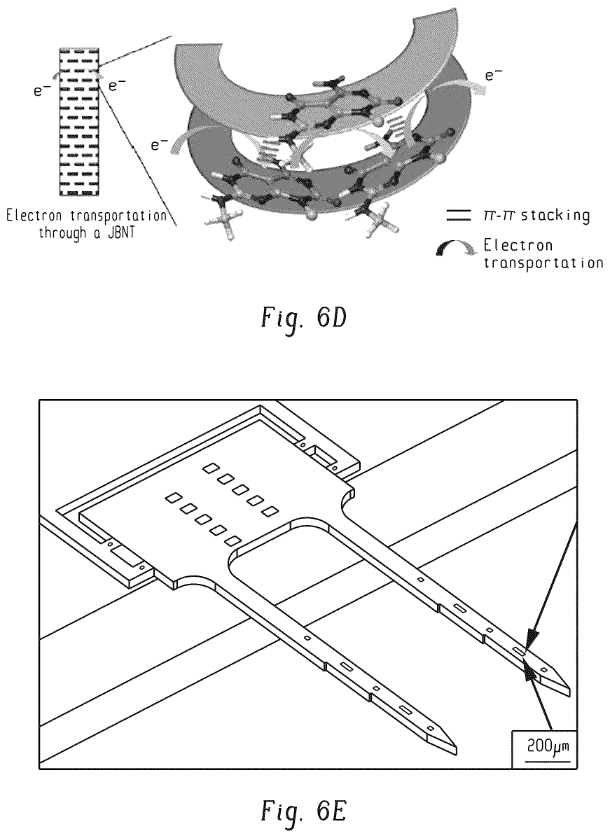

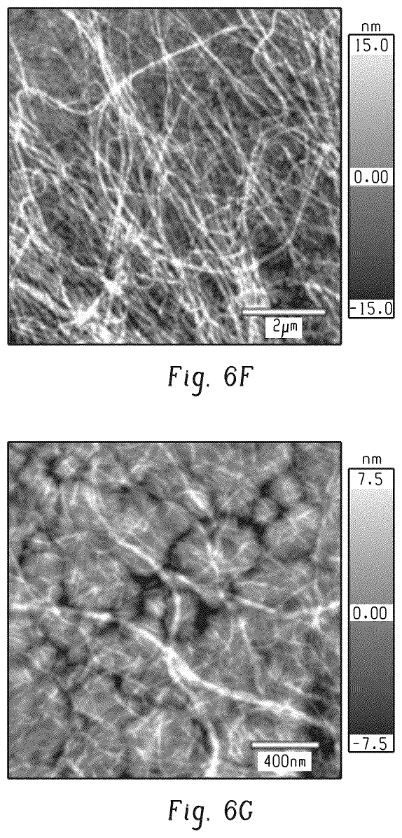

JBNT's DNA-mimicking chemical structure is shown in FIG. 6A. Hydrogen bonding in FIG. 6B facilitates rosette assembly while closely stacked aromatic rings in FIG. 6C and FIG. 6D enable π-π electron transfer. JBNT was prepared at a concentration of 1 mg/mL and deposited onto the two shanks of intracortical microelectrodes. The microelectrode probe design used in this example is shown in FIG. 6E with the shanks constituting the major regions of interest. AFM imaging within the recording/stimulating electrode sites, upper and lower arrows, confirmed that the JBNT arrange horizontally on the microelectrodes (FIG. 6F and FIG. 6G, respectively).

### Electrical Conductivity

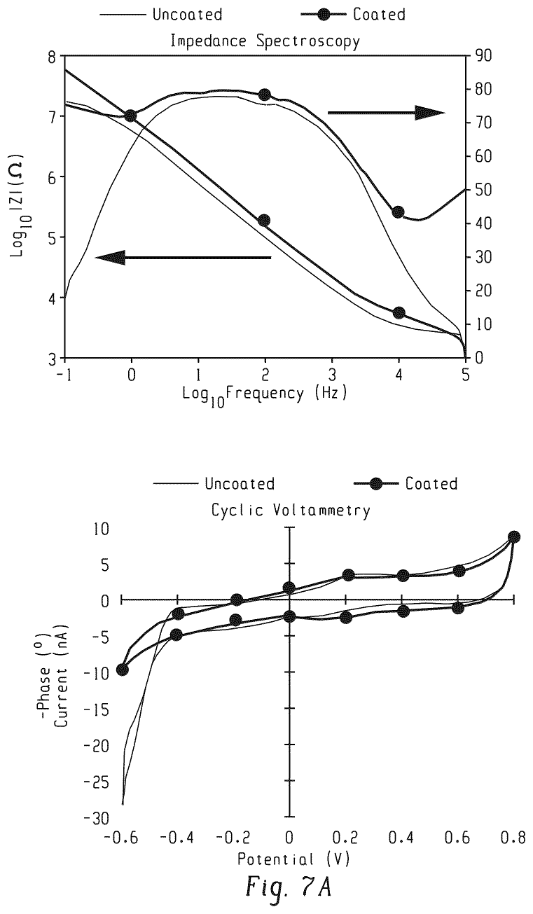

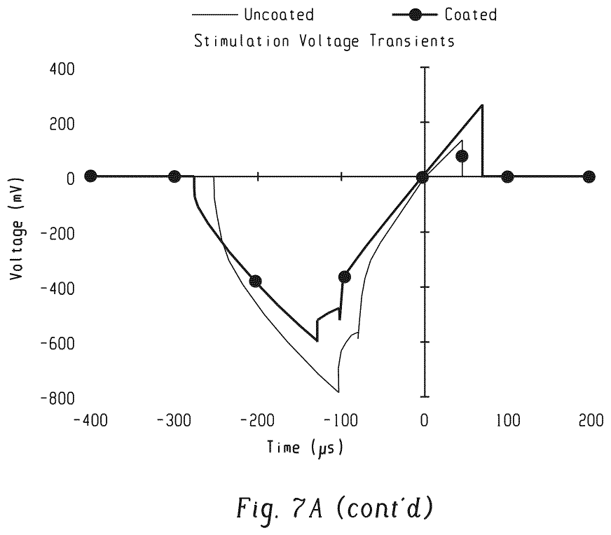

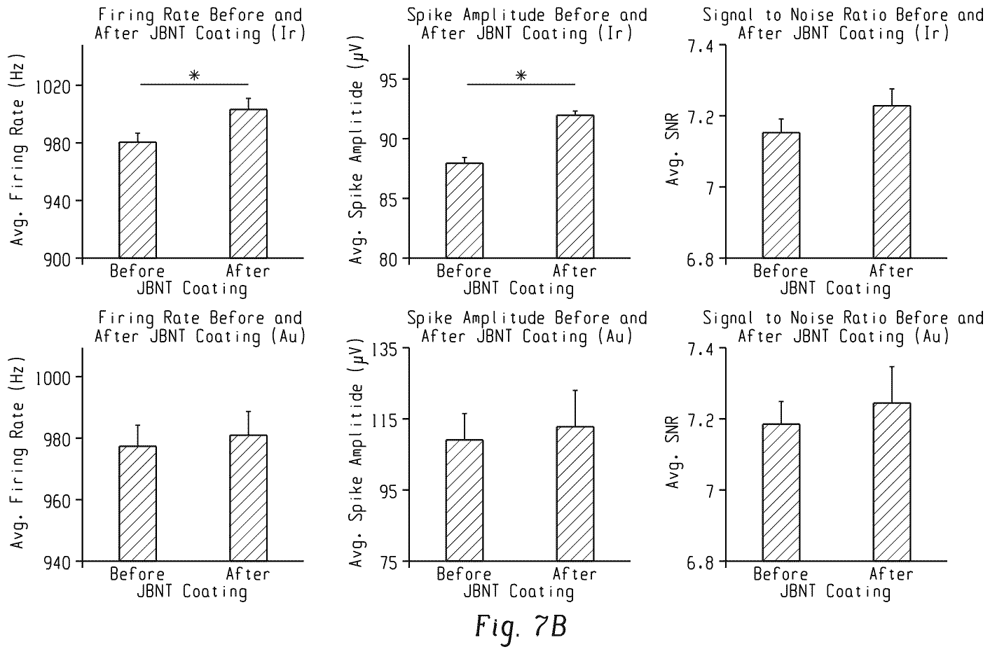

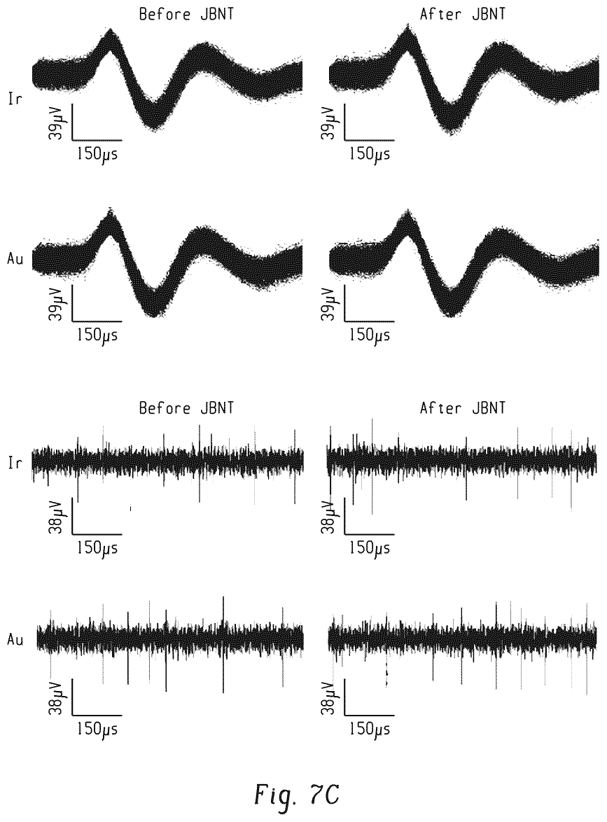

The electrochemical properties of the iridium electrodes with and without JBNT were quantified (FIGS. 7A-7C). Impedance magnitudes were slightly greater and phase angles were slightly more capacitive after coating (FIG. 7A, top). Cyclic voltammograms showed that charge storage capacities were similar but exhibited a reduced hydrogen reduction after JBNT coatings were added (FIG. 7A, middle). In addition, constant-current charge injection pulses produced reduced voltage transients after JBNT coating, which is desired in terms of charge injection capability during neural stimulation (FIG. 7A, bottom).

JBNT coating had minimal impact on neural probes' ability to detect simulated pseudo-neural spike signals in vitro before and after JBNT coating (FIG. 7B and FIG. 7C). Gold microelectrode sites experienced no significant change in measured firing rate, average spike amplitude, or signal-to-noise-ratio (p=0.73, 0.80, and 0.61, respectively), while iridium microelectrode sites recorded slightly higher firing rates (p=0.047) and larger spike amplitudes (p<0.001) after JBNT coating (FIG. 7B). No significant change in signal-to-noise ratio was found in iridium microelectrodes, similarly to gold (p=0.26). Sorted units and high-pass filtered data are illustrated in FIG. 3C for gold and iridium microelectrodes, which were highly similar to each other before and after JBNT coating.

### Neuron Anchorage, Integration, and Functions

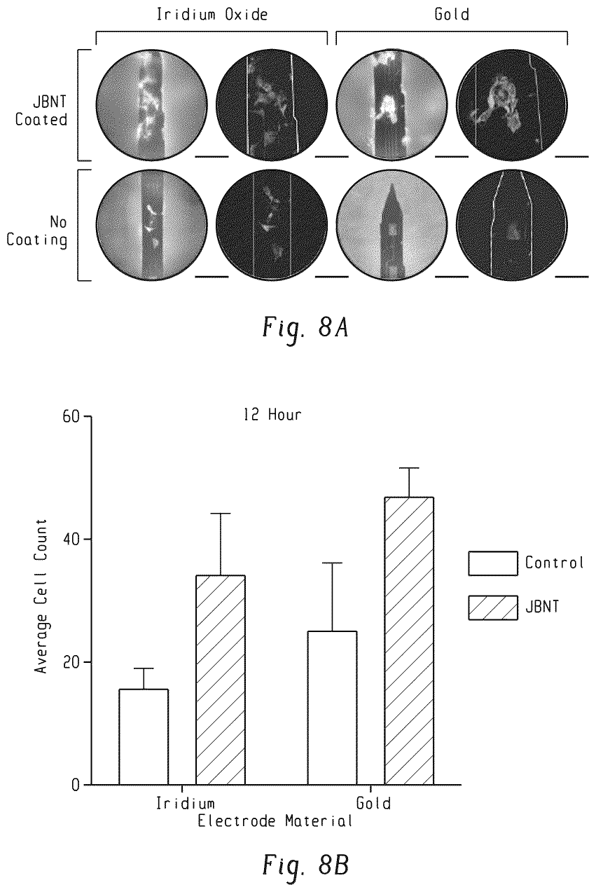

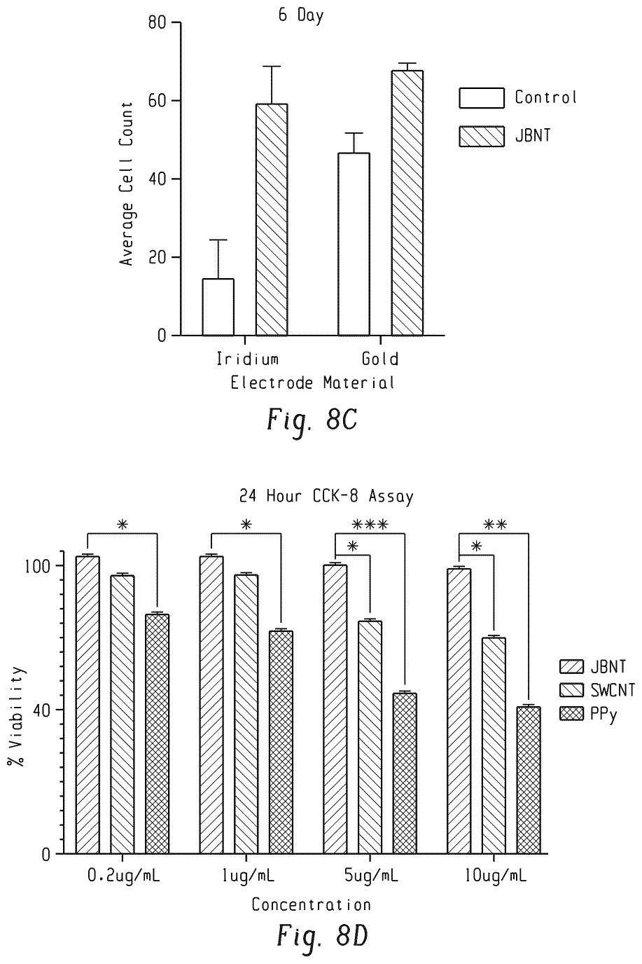

SH-SY5Y cells have previously been used to model neuron cell response to conductive polymer chemistry and probe modifications. SH-SY5Y cells that were cultured on probes for a 6-day acute timeframe were subject to standard differentiation protocol and subsequently analyzed via confocal microscope to study general cellular behavior patterns. FIG. 8A show fluorescent microscope (left) and confocal scan (right) capturing electrode sites after 6-day differentiation of SH-SY5Y cells. Scale bars 100 μm. Fluorescence staining showed distinct favorability to JBNT coated probes versus their non-coated control as seen in FIG. 8A and FIG. 8B. Increased concentrations of cells were observed to be “crowded” around JBNT coated electrodes as opposed to their control counterparts, which demonstrated singular cell activity. Cellular anchorage on and around each electrode site was further analyzed by single cell count. 12-hour and 6-day cultures (respectively FIG. 8B and FIG. 8C) revealed a favorability towards JBNT coated probes, albeit with high variability between groups. Although a general increase in anchorage was hypothesized for JBNT coated groups, the inventors believe that the variability is a direct result of in vitro well size contrasted with extremely small target site for culture. General cell biocompatibility was quantified using two commonly studied electrically conductive polymer coatings: single-wall carbon nanotubes (SWCNTs) and Polypyrrole (PPy). Cell Counting Kit-8 (CCK-8) assay was used to determine the viability of cells after 24-hour incubation with scaled concentrations of each conductive polymer coating material. At lower concentrations (0.2-1 μg/mL), PPy presented significantly reduced cell viability compared to JBNT and SWCNTs. At higher concentrations (5-10 μg/mL), JBNT cultured groups maintained statistically significantly improved cell viability in comparison to both conductive polymers (SWCNTs and PPy) as seen in FIG. 8D. JBNTs biocompatibility can be largely attributed to the biomimetic composition and hydrogen bonding that produce little to no cytotoxic effect even at relatively high concentrations.

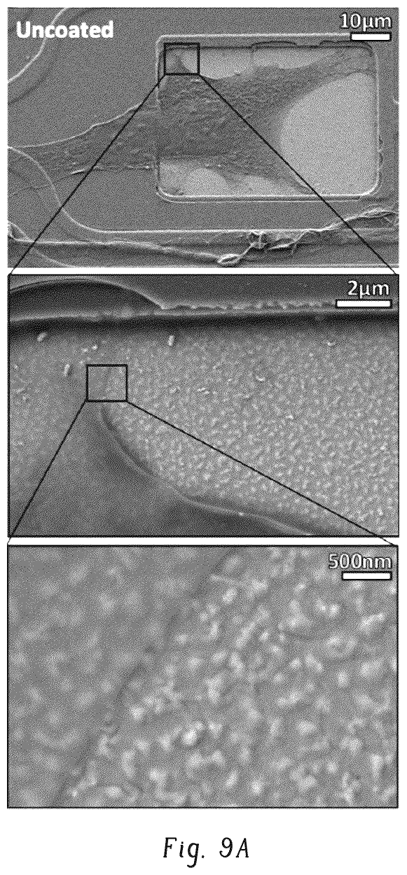

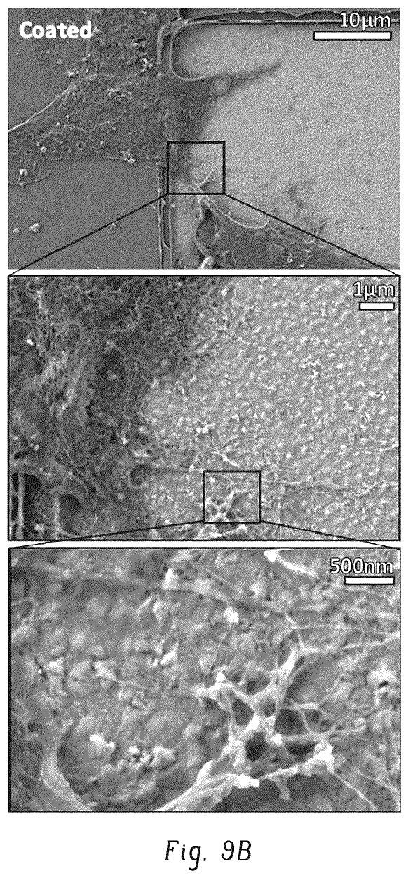

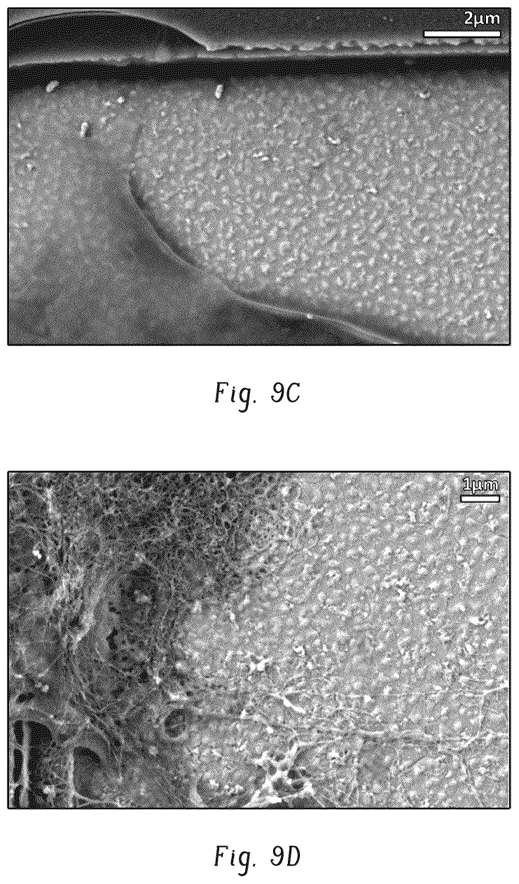

Since JBNT coating behavior and cell favoritism was consistent between iridium and gold electrode probes, SEM was used to look at both probes with respect to cell interface behavior. SEM images were taken to investigate cell-to-electrode interactions as well as neurite advancement onto the JBNT coated substrates fixed after 6-day culture. FIG. 9A demonstrates typical neural cell attachment on a conventional gold electrode substrate with no additional coating at increasing magnification from top to bottom. No cell-to-electrode integration is observed at the nano-scale interface and notable bio-anchorage sites appear at the edge of the electrode well. JBNT coated gold electrodes, on the other hand, showed significant cell-to-electrode interface integration as seen in FIG. 9B. Higher magnification images (increasing magnification from top to bottom) further demonstrated cytoplasm outgrowth into nanotube bundle formations. Color overlays for higher magnification images highlight representative boundaries between patches of anchored cells and the electrode surface (FIG. 9C and FIG. 9D). In these images, it is easy to distinguish the lack of cell-to-electrode integration on the uncoated control (FIG. 9C) and the highly integrated cells on the coated electrode (FIG. 9D).

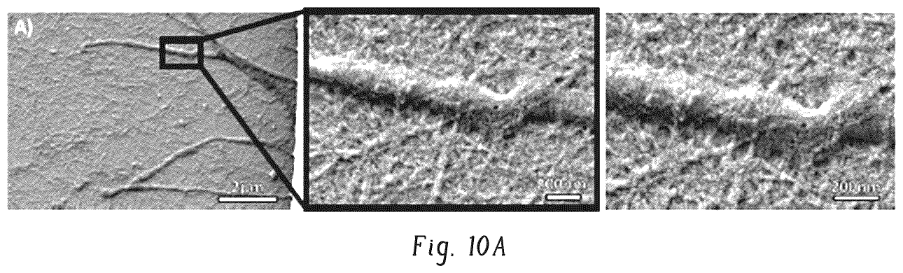

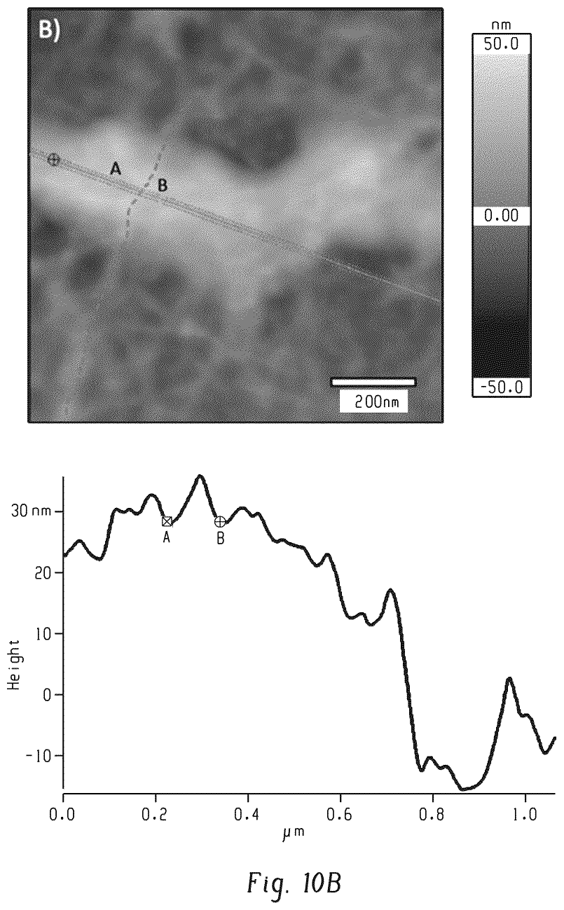

Similar SEM imaging also suggests overgrowth between JBNT bundles and neurite progressions for 6-day cultures as seen in FIG. 10A. Localized AFM scans revealed evidence of bundle formations lying on top of the neurite extensions as seen in FIG. 10B. It is believed that this phenomenon took place during cell proliferation and differentiation as a result of the relatively mechanically compliant biomimetic JBNT bundles.

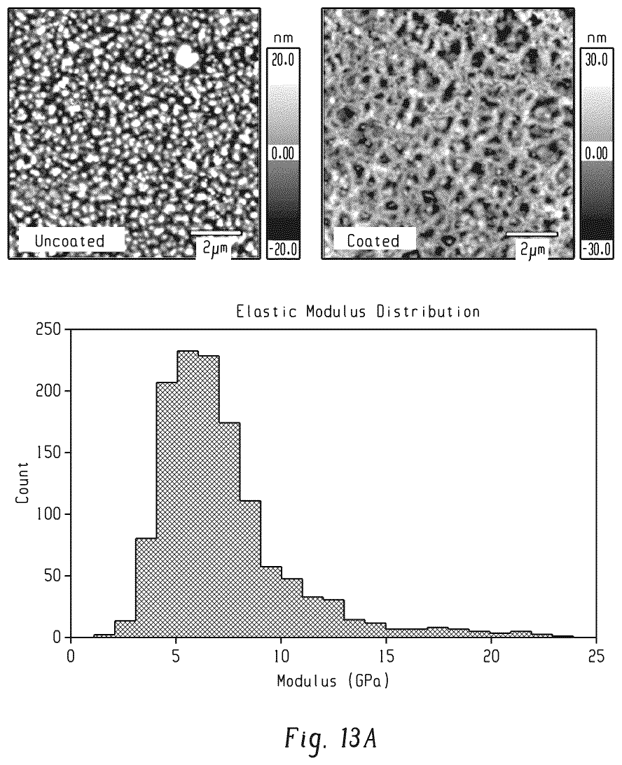

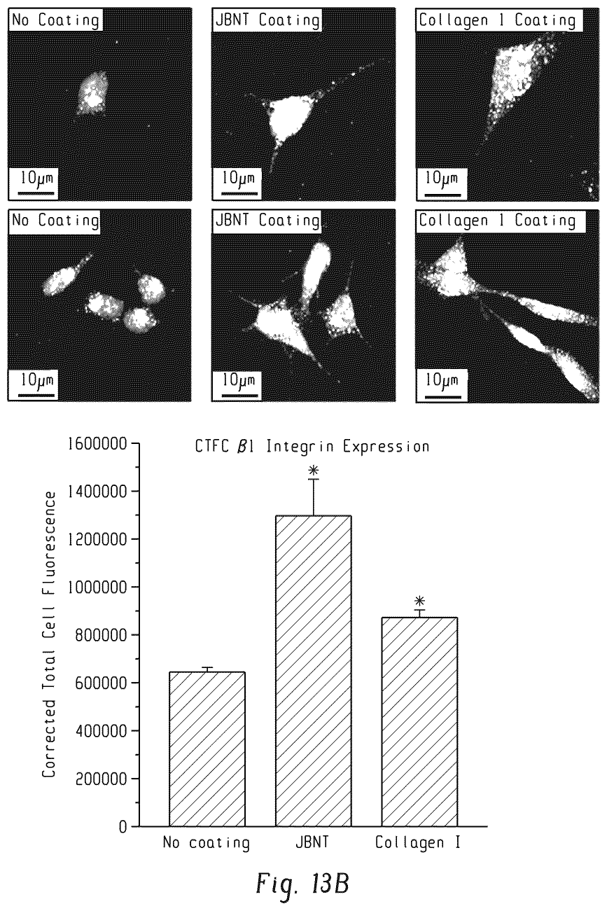

Without being bound by theory, it is believed that due to higher cell anchoring on these optimized probes, interelectrode connections were sometimes observed for JBNT coated devices. These comprise multiple cell connections terminating on respective electrode surfaces. SEM imaging on coated iridium probes cultured long term (14 days) revealed JBNT-integrated anchorage sites remaining after prolonged in vitro culture (FIG. 11). Complimentary AFM imaging on a separate coated iridium probe and respective stiffness values (FIG. 13A) suggests that these dense matrices of JBNT withstand prolonged culture conditions to further enhance cell behavior.

Off-electrode qualitative characterization was conducted to determine the selectivity of JBNTs as the probes are coated and air-dried. Low magnification JBNT SEM and subsequent regions of interest demonstrated significant JBNT aggregation on multiple material surfaces. JBNTs are observed on iridium electrodes as well as the silicon dioxide insulation. Each region of focus investigated the JBNT coating density and behavior at the electrode, insulation, and cell-to-insulation interfaces. Typical cell integration into the JBNT coated substrate was observed, further supporting favorable cell attachment behavior. The control demonstrated typical cell behavior observed in previous negative control groups that included rigid cell-to-substrate termination and reduced bio-anchorage.

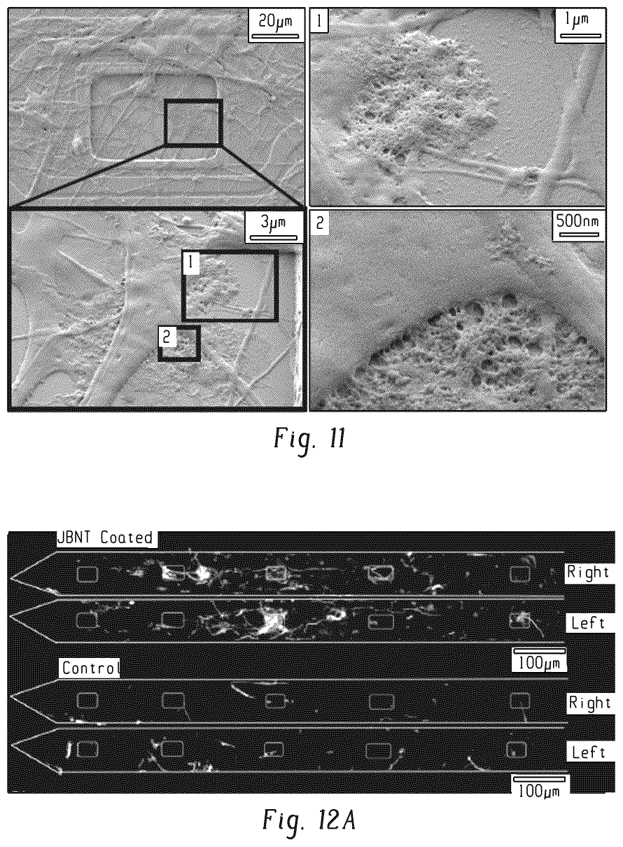

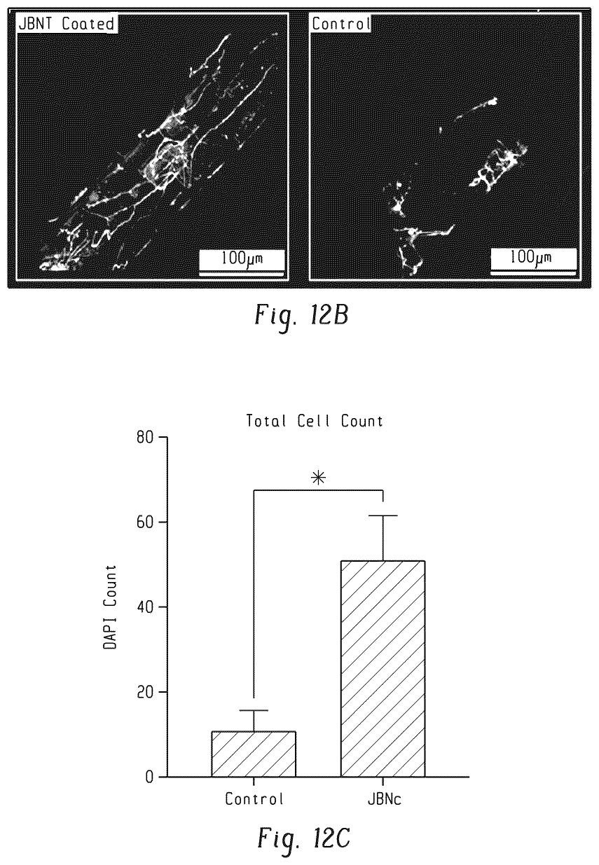

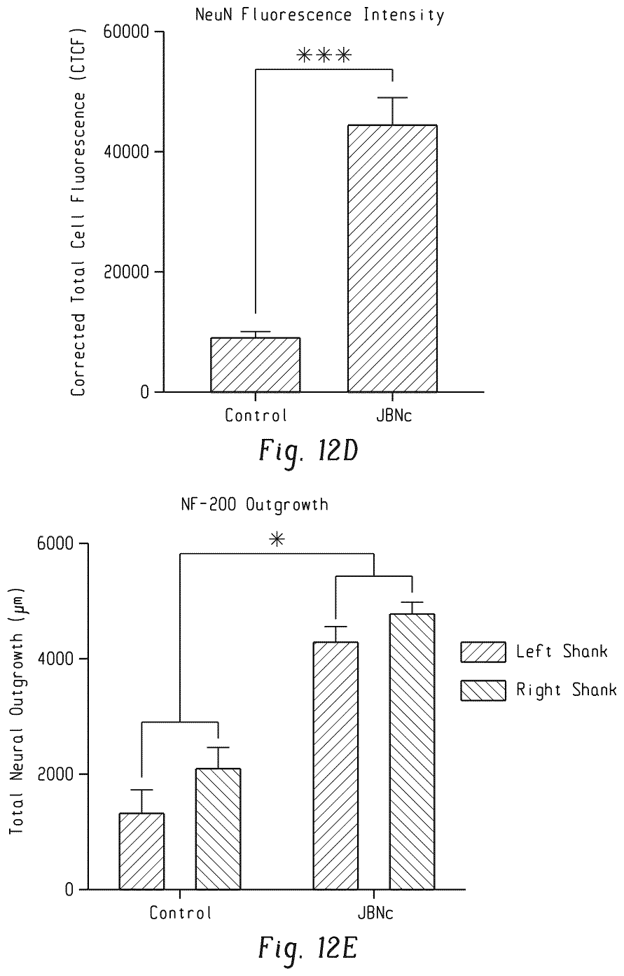

Immunochemical staining revealed significant discrepancies in cell maturation and growth between experimental groups. After two weeks of differentiation culture, sectioned confocal scans were reassembled to show cell coating along the two shanks of each probe (FIG. 12A). Representative sections of NeuN and NF-200 expression are shown in FIG. 12B for comparison. Overall adhesion favoritism was noted by DAPI count which showed a significant difference between groups (FIG. 12C). NeuN expression on neurons cultured on JBNT coated probes expressed significantly higher levels of fluorescence compared to the control group (FIG. 12D). Furthermore, NF-200 staining revealed significantly higher neural outgrowth on JBNT coated probes (FIG. 12E). To mitigate any concern of cell seeding density differences, the area surrounding each probe (left, right, and center of the shanks) were scanned and cell density was analyzed. It was found that although the control shanks demonstrated significantly lower cell count on the regions of interest, the surrounding substrate between and around the control shanks held a significantly higher density of cells in comparison to the surrounding area of the JBNT coated probe shanks. This supports the idea that JBNT coated probes provide a more favorable substrate for cells to adhere to, therefore causing fewer cells to migrate towards the surrounding substrate.

β1 integrin staining was conducted to further characterize adhesion performance in the acute timeframe (FIG. 13B). SH-SY5Y cells were deposited onto silicon dioxide substrates with JBNT and collagen 1 fibril coatings and fixed 8 hours later. Single cell and multi-cell regions demonstrate an increase in β1 integrin expression and focal adhesion production during this short adhesion period. Cell surface area and fluorescence intensity increases significantly after coating, as cells on coated surfaces begin to integrate into favorable substrates. Corrected total cell fluorescence (CTCF) analysis on single cell images showed significant fluorescence signal increase in JBNT and Coll groups.

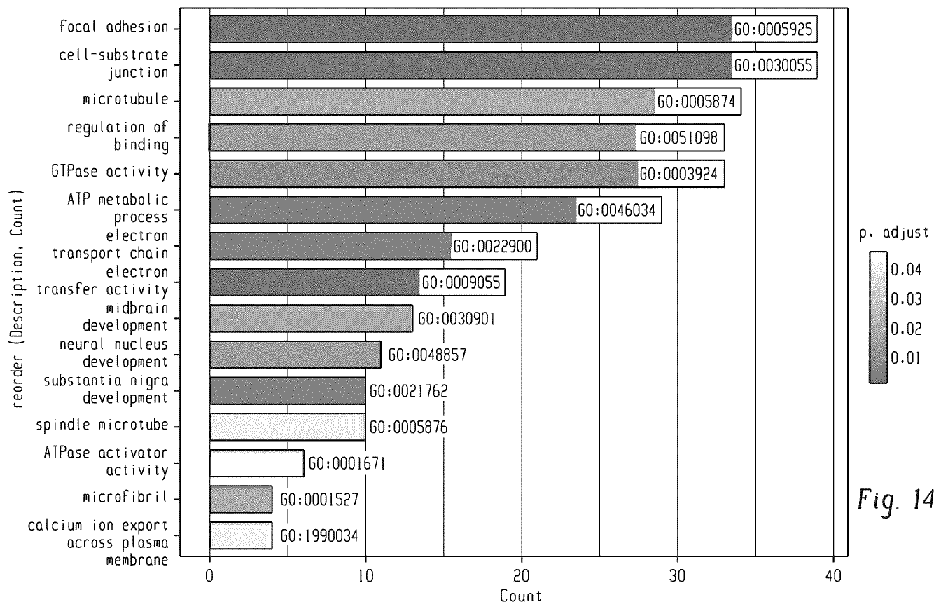

After two weeks of differentiation culture on Janus base nanocoating and control microelectrodes, SH-SY5Y cells were manually isolated from their substrate and lysed immediately for single cell RNA sequencing (scRNA-seq) analysis. Cells were barcoded and pooled prior to illumina sequencing and the neural transcriptome for each individual cell was collected and combined by substrate group for analysis. Following sequence alignment and mapping, cells were grouped based on substrate and analyzed for differentially expressed genes. Differential gene expression was processed in R and analyzed using Gene Ontology (GO)-term analysis for statistically significant and differentially regulated gene families present in cells bound to Janus base nanocoating (FIG. 14). Many noteworthy patterns arose, including the significant regulation of genes governing focal adhesion/cell-substrate junctions and binding. This paralleled a significant upregulation in cytoskeletal remodeling through microtubule and microfibril activity. Neuronal nuclei development was also a significantly upregulated group which contributed to the significant gene counts found within the midbrain and substantia nigra development groups. GTPase activity was also upregulated, particularly with respect to the Rho GTPase family which provides insight into potential mechanisms at play during the differentiation process.

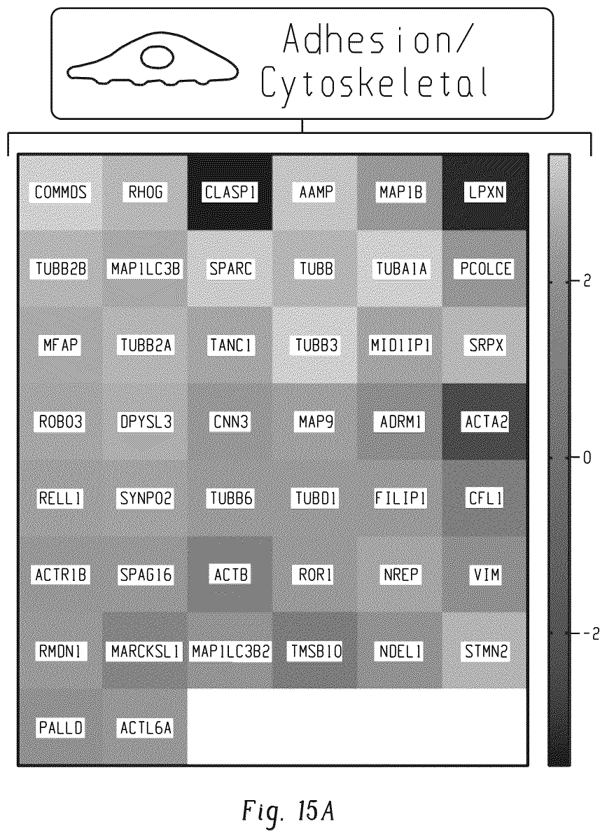

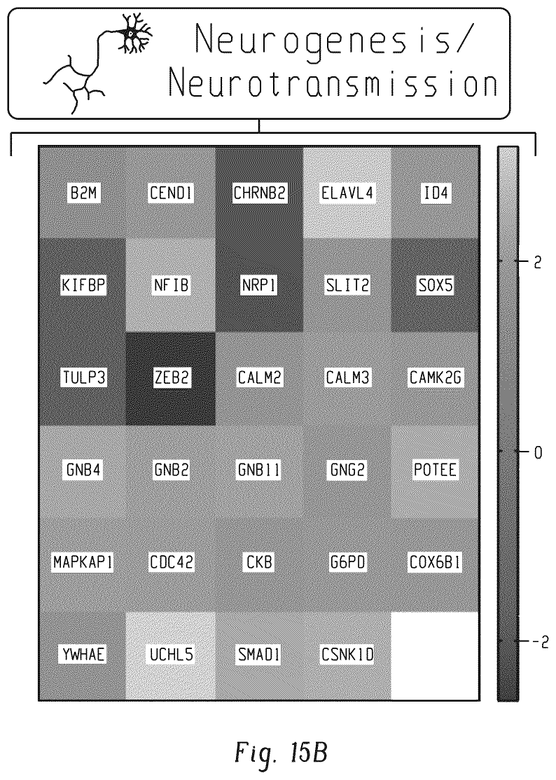

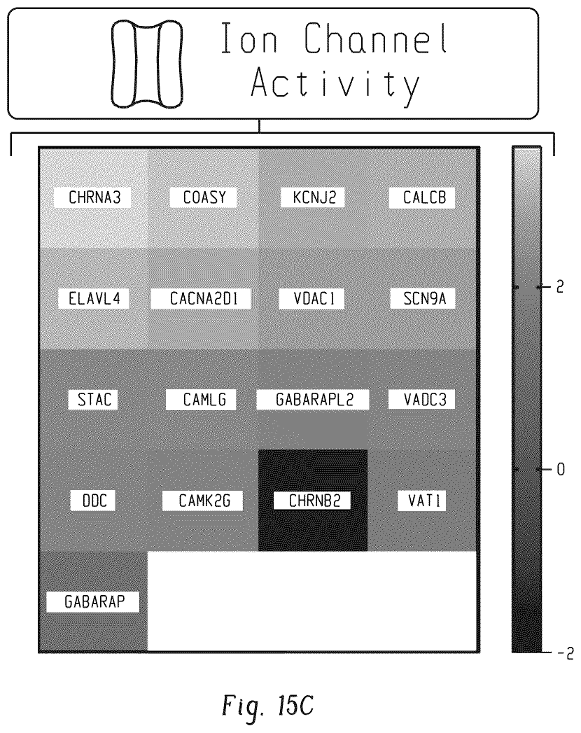

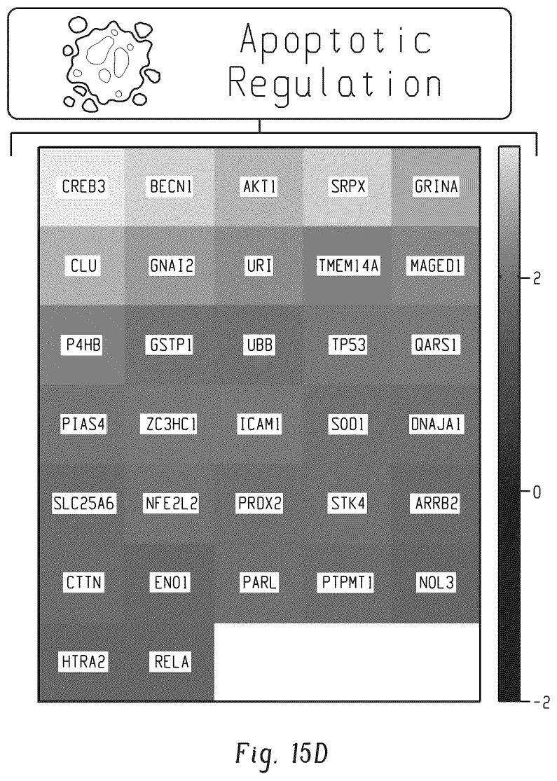

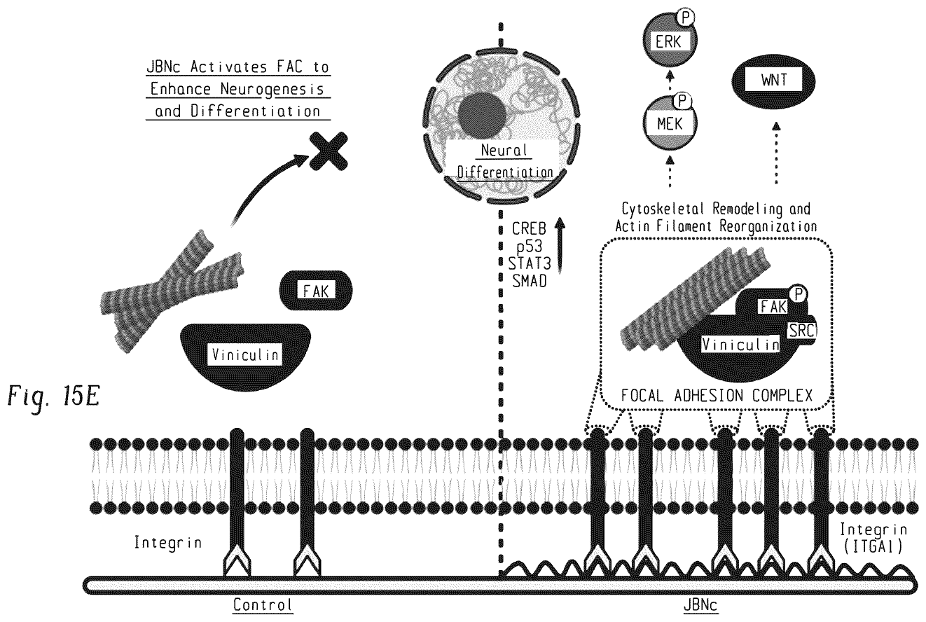

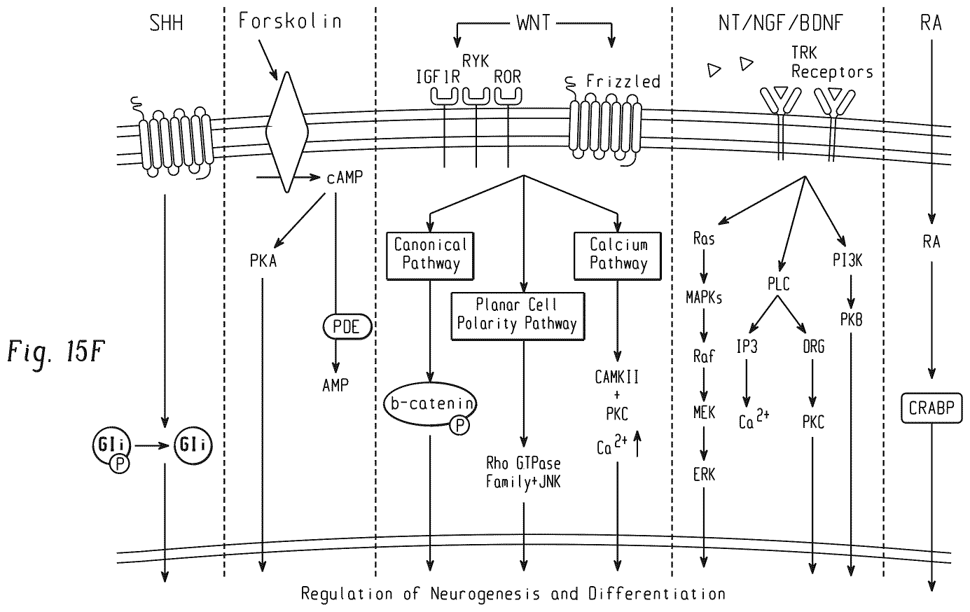

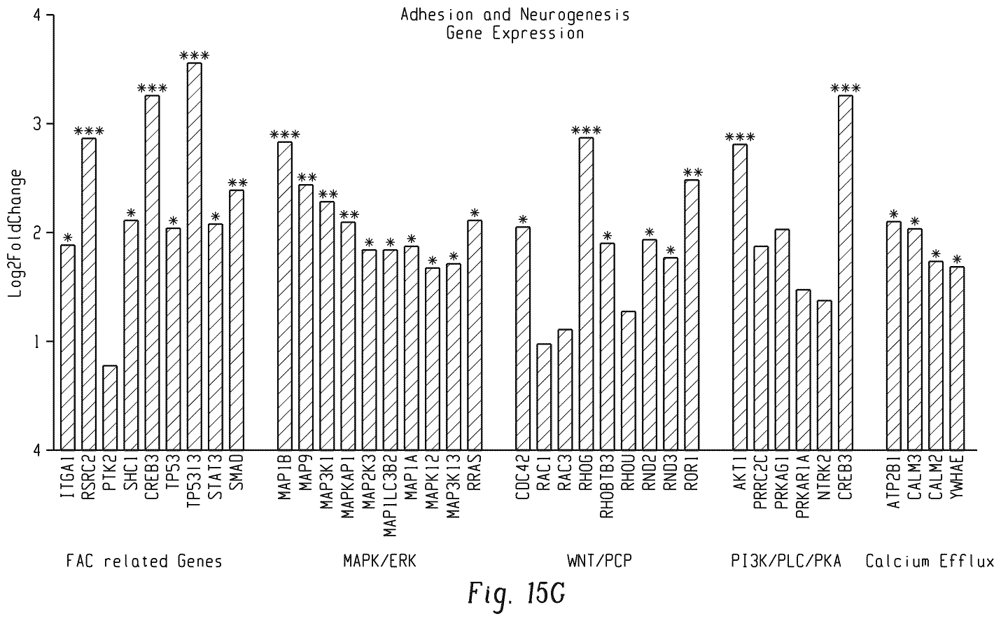

Following GO-term analysis, SH-SY5Y differentiation were assessed using groups of genes commonly modulated during neurogenic maturation including cytoskeletal rearrangement, neurogenesis, ion channel activity, and apoptotic regulation (FIG. 15A, FIG. 15B, FIG. 15C, and FIG. 15D). Based on previous evidence of enhanced differentiation, signaling pathways, and influential transcriptomic complexes directly effecting neurogenic activity were investigated. The evident enhancement of cell-to-substrate binding and subsequent cytoskeletal rearrangement suggested heavy involvement from the Focal Adhesion Complex (FAC) because of Janus base nanocoating interfacing (FIG. 15E). Many genes associated with FAC activity were significantly upregulated in the Janus base nanocoating group, leading to an increase in FAC-dependent neurogenic processes including proliferation and differentiation.

Due to the varied signaling cascades that may influence neural differentiation, pathways like WNT, MAPK/ERK, PI3K/AKT, and others were studied to identify differentially expressed genes in the Janus base nanocoating group (FIG. 15F and FIG. 15G). As a result of increased FAC activity, cells grown on Janus base nanocoating were found to express a variety of genes belonging to the MAPK/ERK pathway, as well as many belonging to the PI3K/AKT cascade. Additionally, cyclic adenosine monophosphate (cAMP) protein kinase and Rho family GTPase activity were significantly upregulated in cells cultured on Janus base nanocoating. Although many differentiation pathways demonstrated differential expression as a result of Janus base nanocoating binding, MAPK/ERK signaling was the most thoroughly activated cascade involved in the enhanced differentiation of SH-SY5Y grown on Janus base nanocoating.

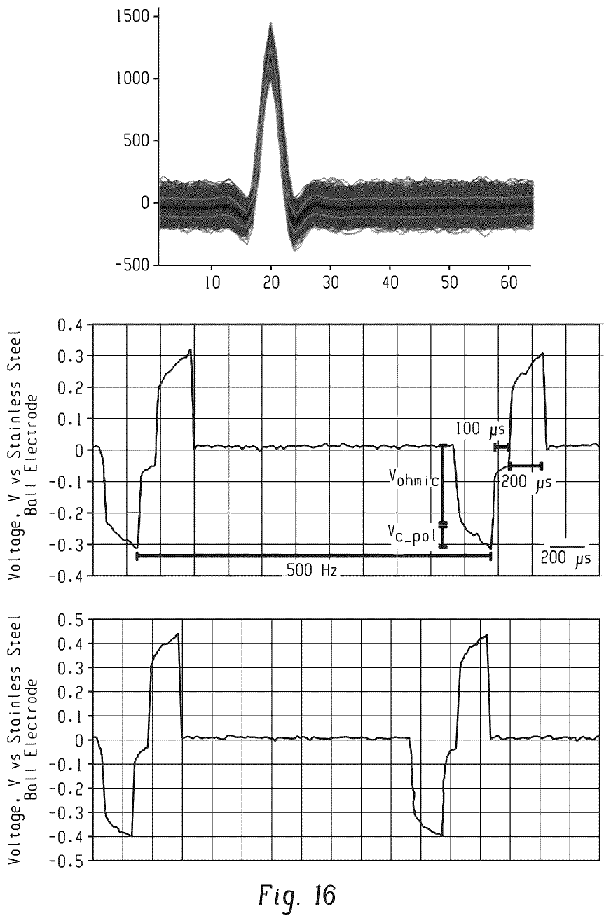

Acute stimulation and recording studies of probes implanted into the rat motor cortex demonstrated successful performance of JBNT nano-coating. Spontaneous neural recording was conducted successfully on non-coated and JBNT-coated probes without any significant difference between groups (FIG. 16).

While preferred embodiments of the present disclosure have been shown and described herein, it will be understood that such embodiments are provided by way of example only. Numerous variations, changes and substitutions will occur to those skilled in the art without departing from the spirit of the present disclosure. Accordingly, it is intended that the appended claims cover all such variations as fall within the spirit and scope of the invention of the present disclosure.

Those skilled in the art will recognize, or be able to ascertain using no more than routine experimentation, many equivalents to the specific embodiments of the present disclosure described herein. Such equivalents are intended to be encompassed by the following claims. It is understood that the detailed examples and embodiments described herein are given by way of example for illustrative purposes only, and are in no way considered to be limiting to the present disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the apparatuses, systems, methods, and processes of the present disclosure will be apparent from the appended claims.

1. An electrode comprising a nanomaterial coating disposed on the electrode, wherein the coating comprises a Janus base nanotube

2. The electrode of claim 1, wherein
the electrode further comprises insulation areas disposed on the electrode;
the nanomaterial coating is a self-assembled nanomaterial coating; or
a combination thereof.

3. The electrode of claim 1, wherein:
(a) the Janus base nanotube comprises a compound of Formula (I): 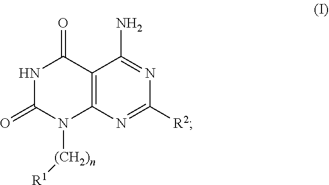
or a pharmaceutically acceptable salt thereof, wherein:
n is 1, 2, 3, 4, 5, or 6;
R¹ is selected from an α-amino acid, a β-amino acid, an α-polypeptide, and a β-polypeptide;
R² is selected from H, CH₃, and NHR^{Z}; and
R^{Z} is independently H or a C₁ to C₂₀ aliphatic group;
(b) the Janus base nanotube comprises a compound of Formula (V): 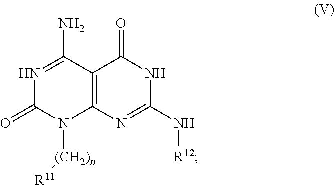
or a pharmaceutically acceptable salt or ester thereof, wherein:
n is 1, 2, 3, 4, 5, or 6;
R¹¹ is selected from an α-amino acid, a β-amino acid, an α-polypeptide, and a β-polypeptide; and
R¹² is H or a C₁ to C₂₀ aliphatic group;
(c) the Janus base nanotube comprises a compound of Formula (VII): 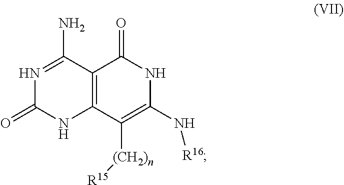
or a pharmaceutically acceptable salt or ester thereof, wherein:
n is 1, 2, 3, 4, 5, or 6;
R¹⁵ is selected from an α-amino acid, a β-amino acid, an α-polypeptide, and a β-polypeptide; and
R¹⁶ is H or a C₁ to C₂₀ aliphatic group; or
(d) a mixture thereof.

4. The electrode of claim 1, wherein:
(a) the Janus base nanotube comprises a compound of Formula (III): 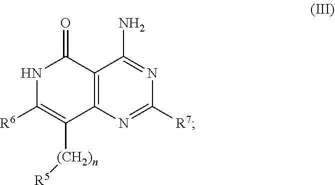
or a pharmaceutically acceptable salt thereof, wherein:
n is 1, 2, 3, 4, 5, or 6;
R⁵ is selected from an α-amino acid, a β-amino acid, an α-polypeptide, and a β-polypeptide; each of R⁶ and R⁷ is independently selected from H, CH₃, and NHR^{Z}; and R^{Z} is independently H or a C₁ to C₂₀ aliphatic group;
(b) the Janus base nanotube comprises a compound of Formula (IX): 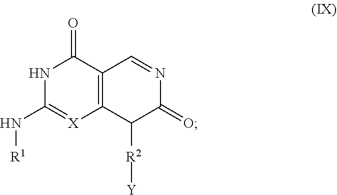
or a pharmaceutically acceptable salt, wherein:
X is CH or nitrogen;
R² is hydrogen or a C₁ to C₂₀ linker group;
Y is absent when R² is hydrogen, or is an amino acid or polypeptide having an amino group covalently bound to an α-carbon of the amino acid and the amino group is covalently bound to the linker group R² when R² is not a hydrogen; and
R¹ is hydrogen or C₁ to C₂₀ aliphatic moiety;
(c) the Janus base nanotube comprises a compound of Formula (X): 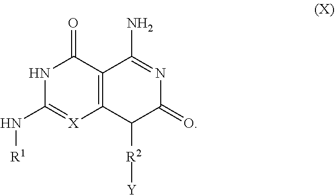
or a pharmaceutically acceptable salt, wherein:
X is CH or nitrogen;
R² is hydrogen or a C₁ to C₂₀ linker group;
Y is absent when R² is hydrogen, or is an amino acid or polypeptide having an amino group covalently bound to an α-carbon of the amino acid and the amino group is covalently bound to the linker group R² when R² is not a hydrogen; and
R¹ is hydrogen or C₁ to C₂₀ aliphatic moiety; or
(d) a mixture thereof.

5. The electrode of claim 1, wherein:
(a) the Janus base nanotube comprises a compound of Formula (II): 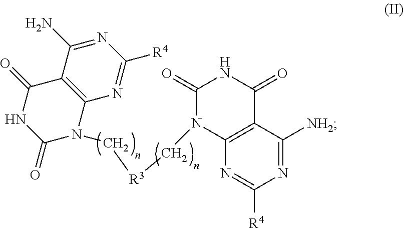
or a pharmaceutically acceptable salt thereof, wherein:
each n is independently 1, 2, 3, 4, 5, or 6;
R³ is selected from an α-amino acid, a β-amino acid, an α-polypeptide, and a β-polypeptide; each of R⁴ is independently H, CH₃, or NHR^{Z}; and
each R_{Z} is H or a C₁ to C₂₀ aliphatic group;
(b) the Janus base nanotube comprises a compound of Formula (IV): 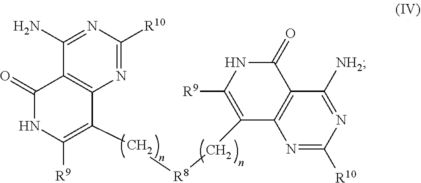
or a pharmaceutically acceptable salt thereof, wherein
each n is independently 1, 2, 3, 4, 5, or 6;
R⁸ is selected from an α-amino acid, a β-amino acid, an α-polypeptide, and a β-polypeptide; each R⁹ and R¹⁰ is independently H, CH₃, or NHR^{Z}; and
each R^{Z} is H or a C₁ to C₂₀ aliphatic group;
(c) the Janus base nanotube comprises a compound of Formula (VI): 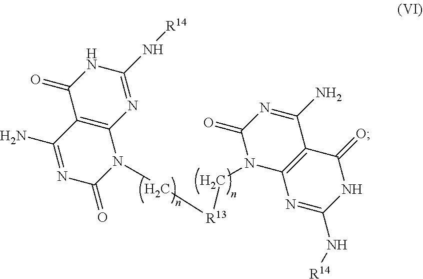
or a pharmaceutically acceptable salt thereof, wherein
each n is independently 1, 2, 3, 4, 5, or 6;
R¹³ is selected from an α-amino acid, a β-amino acid, an α-polypeptide, and a β-polypeptide; and
each R¹⁴ is independently H or a C₁ to C₂₀ aliphatic group;
(d) the Janus base nanotube comprises a compound of Formula (VIII): 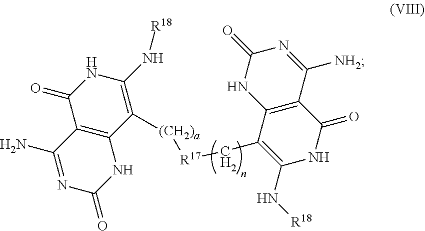
or a pharmaceutically acceptable salt thereof, wherein:
each n is independently 1, 2, 3, 4, 5, or 6;
R¹⁷ is selected from an α-amino acid, a β-amino acid, an α-polypeptide, and a β-polypeptide; and
each R¹⁸ is independently H or a C₁ to C₂₀ aliphatic group; or
(e) the Janus base nanotube comprises a compound of Formula (XI): 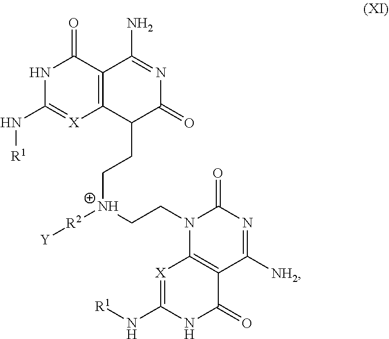
or a pharmaceutically acceptable salt, wherein:
each X is independently CH or nitrogen;
R² is hydrogen or a C₁ to C₂₀ linker group;
Y is absent when R² is hydrogen, or is an amino acid or polypeptide having an amino group covalently bound to an α-carbon of the amino acid and the amino group is covalently bound to the linker group R² when R² is not hydrogen; and
each R¹ is independently hydrogen or a C₁ to C₂₀ aliphatic moiety; or
(f) a mixture thereof.

6. (canceled)

7. The electrode of claim 1, wherein the nanomaterial coating further comprises one or more therapeutic molecules.

8. The electrode of claim 7, wherein the one or more therapeutic molecules are selected from TGFβ, VEGF, IGF, EGF, PDGF, BMPs, FGF, GDNF, HGF, PGF, NGF, TNF-α, SDF-1, dexamethasone, resveratrol, a small interfering ribonucleic acid (siRNA), a micro ribonucleic acid (miRNA), a growth factor, a small-molecule drug, and a mixture thereof.

9. The electrode of claim 1, wherein the nanomaterial coating is a single compartment nanomaterial coating.

10. The electrode of claim 1, wherein the nanomaterial coating is a multiple compartment nanomaterial coating.

11. An apparatus comprising the electrode of claim 1.

12. A method comprising coating an electrode with Janus base nanotubes to create the electrode of claim 1.

13. The method of claim 12, wherein
coating comprises depositing the Janus base nanotubes on to the surface of the electrode by any appropriate coating method;
coating or depositing includes incubating the electrode in a solution comprising Janus base nanotubes;
coating or depositing includes incubating the electrode in a solution comprising Janus base nanotubes at a concentration of about 0.001 mg/mL to about 10.0 mg/mL; or
a combination thereof.

14. (canceled)

15. The method of claim 13, wherein the solution further comprises a liquid selected from water, an organic solvent and a buffer.

16. The method of claim 14, wherein
coating comprises drying for a sufficient time to dry the solution;
the solution further comprises one or more therapeutic molecules; or
a combination thereof.

17. (canceled)

18. The method of claim 16, wherein the therapeutic molecules are selected from TGFβ, VEGF, IGF, EGF, PDGF, BMPs, FGF, GDNF, HGF, PGF, NGF, TNF-α, SDF-1, dexamethasone, resveratrol, a small interfering ribonucleic acid (siRNA), a micro ribonucleic acid (miRNA), a growth factor, a small-molecule drug, and a mixture thereof.

19. The method of claim 12, wherein
the method further comprises air-drying, heated-drying, lyophilizing, electrospinning, dipping, or a combination thereof;
coating and/or depositing is repeated; or
a combination thereof.

20. (canceled)

21. A method or device for sensing biological activity, comprising:
placing the electrode of claim 1, or an apparatus comprising the electrode, proximate to biological tissue or muscle; and
monitoring a signal on the electrode or electrically stimulating biological tissue or muscle by way of a wire connected to the electrode,
wherein the signal is indicative of the biological activity.

22. A method of stimulating biological tissue, promoting cell adhesion, inhibiting inflammation, or promoting growth, functions and/or neurogenesis of a neural cell(s) and tissue(s), the method comprising:
placing the electrode (e.g., microelectrode or macroelectrode) of claim 1, or an apparatus comprising the electrode, proximate to biological tissue; and
applying a current and/or a voltage to the biological tissue with the electrode, stimulating the biological tissue, promoting cell adhesion, inhibiting inflammation, or promoting growth, functions and/or neurogenesis of a neural cell(s) and tissue(s).

23. (canceled)

24. (canceled)

25. (canceled)

26. The method of claim 21, wherein the biological tissue is brain tissue, heart, muscle, spine tissue, spinal cord tissue, nerve tissue, eye tissue, ear tissue, bone tissue, bone marrow tissue, joint tissue, liver tissue, kidney tissue, lung tissue, bladder tissue, or intestine tissue.

27. The method of claim 22, wherein the biological tissue is brain tissue, heart, muscle, spine tissue, spinal cord tissue, nerve tissue, eye tissue, ear tissue, bone tissue, bone marrow tissue, joint tissue, liver tissue, kidney tissue, lung tissue, bladder tissue, or intestine tissue.
